(19) United States
(12) Patent Application Publication
(10) Pub. No.: US 2023/0134637 A1
(43) Pub. Date: May 4, 2023

(54) METHODS, DEVICES AND SYSTEMS FOR PROVIDING STIMULUS TO GUIDE MOVEMENT

(71) Applicant: Neuroscience Research Australia (NeuRA), Randwick, New South Wales (AU)

(72) Inventors: Andrew Matthew Dalhousie BRODIE, Randwick, New South Wales (AU); Kim DELBAERE, Randwick, New South Wales (AU)

(21) Appl. No.: 17/767,937

(22) PCT Filed: Oct. 9, 2020
(86) PCT No.: PCT/AU2020/051088

§ 371 Date: Apr. 11, 2022

(30) Foreign Application Priority Data
Oct. 11, 2019 (AU) 2019903826

(51) Int. Cl. A61N 1/36; A61B 5/11; A61N 1/04

(52) U.S. Cl. CPC A61N 1/36003; A61B 5/112; A61B 5/1101; A61N 1/36031; A61N 1/0484; A61B 2562/0219

(57) ABSTRACT

A stimulation device for guiding movement of at least part of a body, the device comprising: at least one body-fixed stimulator, configured to provide stimulus to the part of the body; at least one control unit in communication with the at least one body-fixed stimulator, the control unit configured to generate a control signal to control the at least one body-fixed stimulator. Embodiments of the device or system may be configured perform a method of guiding movement of at least part of a body comprising the steps of: comparing at least one parameter relating to a movement with at least one pre-defined criteria; generating a control signal from at least one control unit if the at least one parameter does not meet the pre-defined criteria; and providing a stimulus to the body in response to the control signal through at least one body-fixed stimulator to guide the movement.

### FIELD OF THE INVENTION

The present invention relates to methods and devices for providing stimulus. More particularly, the invention relates to methods and devices for providing stimulus to guide movement of at least part of a body.

The invention has been developed primarily for use in guiding movement to achieve a target movement or reduce undesirable movement by providing stimulus to a limb of a body. While some embodiments will be described herein with particular reference to that application, it will be appreciated that the invention is not limited to such a field of use and is applicable in broader contexts.

### BACKGROUND

The following discussion of the prior art is intended to facilitate an understanding of the invention and to enable the advantages of it to be more fully understood. It should be appreciated, however, that any reference to prior art throughout the specification in no way be considered as an admission that such art is widely known or forms part of common general knowledge in the field.

Guiding and improving movement of a limb of a body is particularly useful in assisting those who suffer from ailments which result in limited movement or undesirable movement, such as tremors. For example, those who have suffered from a stroke may find it difficult to mobilise muscles in a limb. Additionally, for example, those who suffer from Parkinson's disease may find it difficult to walk in a stable manner, suffer from freezing-of-gait (FOG) and/or suffer from limb tremor.

Research into this area investigates the effect of rhythmic auditory and/or attentional cues to assist people with Parkinson's disease, including the use of vibrational motors. Vibrating motors have been used in several research papers in people with Parkinson's disease. However, the vibrating motors are generally very large and are not precisely positioned or coordinated to optimise transfer of information between the central nervous system and peripheral nervous system of the body.

Continuous vibration (e.g. for several seconds), which is vibration to a muscle or part of the body (feet) that is constant, has also been investigated. It has shown limited potential to reduce freezing-of-gait and improve walking ability. Vibrating platforms have also been used as a training device to build muscle mass and may reduce postural sway when someone stands on them. However, continuous vibration may alter proprioception, or increase the excitability of the muscle fibres, but it does not specifically guide the timing or amplitude of target movements.

The application of a single vibration motor, rhythmic, intermittent vibration or one-off vibration pulses has also been tested. In most of these cases, a single vibrating motor is attached to the wrist, and vibrations are repeated using a cadence usually within +−10% of usual cadence. This has shown to be of limited value, and research suggests that it works no better than auditory cues. This is shown, for example, in “The attentional cost of external rhythmical cues and their impact on gait in Parkinson's disease: effect of cue modality and task complexity” (2007) J Neural Transm (Vienna), 114, 1243-8. by Rochester et al. The mechanism of a single vibrating motor is used as a substitute for external auditory cue. Location of the vibration is not considered to be important, and vibrators may be applied at the wrist of a user. The purpose of the research is to set an external cadence that the user must follow. However, the user is required to concentrate on following this external cue, and it is not an automated process.

U.S. patent application Ser. No. 15/638,115 discloses a wearable device comprising a plurality of actuators. The actuators in the wearable device are adjustable relative to one another in terms of their position and in various examples, the actuators may be adjustable relative to one another in terms of their duty cycle, power and/or position based on sensor data. The invention does not disclose a network of multiple coordinated stimulators and sensors that stimulate the peripheral nervous system at multiple locations on the body to optimise and synchronise the two-way transfer of information between the peripheral nervous system and central nervous system and guide movement.

U.S. Pat. No. 9,943,250 discloses a method and system for provoking gait disorders, such as freezing of gait. The invention discloses display of situations calculated to cause freezing of gait which are presented to a subject and identifying incipit freezing of gait using changes in gait parameters. The invention discloses a portable device which detects incipit freezing of gait only.

U.S. Pat. No. 10,653,352 discloses a system comprising several vibration motors attached to the ankle using a plastic housing and insole system. The vibration motors are connected to through a controller to pressure sensors in the insole. The system is designed for people with minimal sensation in their feet. Applying weight to different pressure sensors in the insole causes different vibration motors at the ankle to activate. This system is limited to providing feedback at the ankle that is a direct result of pressure switches in the insole. The invention does not disclose a network of multiple coordinated stimulators (the stimulation applied to the left ankle and right ankle are not coordinated but act independently). The invention does not disclose two-way transfer of information between the peripheral nervous system and central nervous system to guide movement. Rather, it provides only feedback about foot current loading for people who have little or no foot sensation.

U.S. Pat. No. 8,925,392 discloses a system of incorporating pressures sensors into fabric such as smart socks. The sensors are proposed to provide real-time feedback to users. However, it does not disclose using vibrational or electrical stimulation to guide human movement.

Other known preventative measures for freezing-of-gait including the use of walking sticks, lasers projections on the floor, walkers and other devices. However, these measures necessarily require concentration and manual operation by the user and are not automated processes.

It is an object of the present invention to overcome or ameliorate at least one of the disadvantages of the prior art, or to provide a useful alternative.

### SUMMARY OF THE INVENTION

According to a first aspect of the invention, there is provided a stimulation device for guiding movement of at least part of a body, the device comprising:
at least one body-fixed stimulator, configured to provide stimulus to the part of the body;
at least one control unit in communication with the at least one body-fixed stimulator, the control unit configured to generate a control signal to control the at least one body-fixed stimulator.

Preferably, the stimulation device includes a plurality of body-fixed stimulators. More preferably, each of the plurality of body-fixed stimulators are disposed in a predefined location relative to the part of the body.

Preferably, the control signal is generated when at least one pre-defined criteria is met. In a further embodiment, the device comprises at least one sensor in communication with the at least one control unit, the at least one sensor configured to receive monitoring data. Preferably, the control signal is generated on the basis of the monitoring data. In another embodiment, the stimulation device comprises a plurality of sensors.

Preferably, guiding movement of at least part of a body includes directing the part of the body to achieve a target movement or position. More preferably, guiding movement includes directing the body to increase desirable movement and/or decrease undesirable movement. In some embodiments, desirable movements may include but are not limited to walking, running, dancing, standing upright, maintaining postural balance, undertaking postural transitions, reaching, coordinated hand and arm movements and generally moving in a relatively improved manner. In other embodiments, unwanted movements are generally higher frequency oscillations as detected by Fast Fourier Analysis (for tremor), larger involuntary movements as detected by pattern recognition tools (for dyskinesia), excessive postural sway as detected by wearable devices (for gait, standing and postural transitions), excessive step time variability (for gait), low step height and excessively high cadence (for gait festination).

Preferably, the target movement or position is stable gait. In another embodiment, the target movement or position is reduced limb tremor.

In further embodiments, the at least one sensor is fixed to the body by way of integration with a textile. Preferably, the textile forms at least part of a wearable garment. More preferably, the wearable garment is in the form of a sock.

In one embodiment, pre-defined criteria may include at least one of manual or automated operation. Preferably, automated operation includes generating or optimising the control signal using monitoring data from the plurality of sensors that detect key movement parameters. Preferably, key movement parameters include but are not limited to cadence, step length, step-time variability, higher frequency tremor and larger scale unwanted movements.

In accordance with a second aspect of the invention, there is provided a method of guiding movement of at least part of a body comprising the steps of:
(a) comparing at least one parameter relating to a movement with at least one pre-defined criteria;
(b) generating a control signal from at least one control unit if the at least one parameter does not meet the pre-defined criteria; and
(c) providing a stimulus to the body in response to the control signal through at least one body-fixed stimulator to guide the movement.

Preferably, the method further comprises the step of repeating steps (a) to (c) until the at least one parameter meets the at least one pre-defined criteria.

In accordance with third aspect of the invention, there is provide a method of guiding movement of at least part of a body comprising the steps of:
(a) providing a stimulus to the body through at least one body-fixed stimulator to guide a movement;
(b) comparing at least one parameter relating to the movement with at least one pre-defined criteria;
(c) providing further stimulus to the body if the at least one parameter does not meet the pre-defined criteria; and
(d) ceasing stimulation if the parameter meets the pre-defined criteria.

Preferably, the method further comprises the step of measuring the at least one parameter using at least one sensor to receive monitoring data related to the at least one parameter. More preferably, the method further comprises the step of updating at least one stimulation setting based on the received monitoring data to define at least one updated stimulation setting.

In one embodiment, the method further comprises the step of generating an optimised control signal. Preferably, the optimised control signal is generated by using at least the updated stimulation setting. More preferably, the step of generating an optimised control signal comprises using historic movement data to set a range of predetermined optimum criteria and generating the optimised control signal to control within the range of predetermined optimum criteria.

In one embodiment, the method further comprises the step of updating the at least one pre-defined criteria based on the received monitoring data to define at least one updated criterion. Preferably, the optimised control signal is generated by using at least the updated criteria.

In a further embodiment the guiding movement of at least part of a body includes directing the part of the body to achieve a target movement or position. Preferably, the guiding movement includes directing the body to increase desirable movement and/or decrease undesirable movement. In one embodiment, the target movement or position is stable gait. In another embodiment, the target movement or position is reduced limb tremor.

In one embodiment, the at least one sensor is fixed to the body by way of integration with a textile. Preferably, the textile forms at least part of a wearable garment. More preferably, the wearable garment is in the form of a sock.

According to a fourth aspect of the invention, there is provided a stimulation device, configured to perform the method according to the second or third aspect of the invention.

Preferably, the method uses coordinated low-level stimuli thereby to reinforce target neuromuscular signals guiding muscle contractions. This advantageously results in target movements that are relatively more coordinated within and between limbs and repetitions for the user.

Preferably, the method uses coordinated low-level stimuli thereby to dilute unwanted neuromuscular signals in the peripheral nervous system and enable improved feedback about the target movement to the central nervous system. This advantageously reduces the cognitive load required by the user to achieve the target movement.

Advantageously, the method optimises and synchronises the two-way information transfer between the central nervous system and peripheral nervous system. Another advantageous aspect of the invention provides a network of stimulators to provide coordinated stimulation to the peripheral nervous system.

Preferably, the method may be used in combination with pharmacological enhancement, deep brain stimulation, and electrical or magnetic transcortical stimulation, attentional cues and emotional cues that stimulate the central nervous system.

In a further embodiment, there is provided a stimulation device for guiding movement of at least part of a body, the device comprising:
at least one body-fixed stimulator disposed in a location relative to the part of the body, configured to provide stimulus to the part of the body;
at least one control unit in communication with the at least one body-fixed stimulator, the control unit configured to generate a control signal to control the at least one body-fixed stimulator; and
at least one sensor in communication with the at least one control unit, the at least one sensor configured to receive monitoring data, wherein the control signal is generated on the basis of the monitoring data.

In a further embodiment, there is provided a method of guiding movement of at least part of a body comprising the steps of:
(a) providing a stimulus to the body through at least one body-fixed stimulator to guide a movement;
(b) measuring the at least one parameter using at least one sensor to receive monitoring data related to the at least one parameter
(c) comparing at least one parameter relating to the movement with at least one pre-defined criteria;
(d) providing further stimulus to the body if the at least one parameter does not meet the pre-defined criteria; and
(e) ceasing stimulation if the parameter meets the pre-defined criteria.

In accordance with a further embodiment of the invention, there is provided a method of optimising the stimulation device according to claims 1 to 14 for a user, the method comprising the steps of:
(a) repeating a target movement;
(b) switching on a body-fixed stimulator;
(c) obtaining monitoring data;
(d) comparing at least one parameter relating to the monitoring data with historical data for the same target movement; and
(e) determining the most likely search path towards the optimum stimulation settings.

Preferably, optimising the stimulation device is used to determine at least one of the optimum number, type and/or location of stimulators, or at least one of the intensity, frequency and/or duty cycle of stimulation.

### BRIEF DESCRIPTION OF THE DRAWINGS

Preferred embodiments of the disclosure will now be described, by way of example only, with reference to the accompanying drawings in which:

FIG. 1 is a concept diagram of reinforcing target movements;

FIG. 2 is a concept diagram of reducing unwanted movement variability;

FIG. 3 is a diagram of a smart sock embodiment according to the present invention, showing different stimulation locations including under the foot arch and slightly superior to the lateral and medial malleoli;

FIG. 4 is a diagram of a smart glove embodiment according to the present invention;

FIG. 5 is a flowchart diagram showing a method of manual operation of the system;

FIG. 6 is a flowchart diagram showing a method of automatic operation (start/stop stimulation) when unstable gait is detected;

FIG. 7 is a flowchart diagram showing a method of tremor reduction of a limb;

FIG. 8 is a flowchart diagram showing a method of training progression;

FIG. 9 is a flowchart diagram showing a method according to one embodiment of the invention;

FIG. 10 is a flowchart diagram showing a method according to another embodiment of the invention

FIG. 11 is an enlarged perspective view of a smart sock embodiment;

FIG. 12 is a perspective of the smart sock embodiment of FIG. 11;

FIG. 13 is a cross sectional view of a stimulator within the smart sock embodiment of FIG. 11;

FIG. 14 is a perspective view of the stimulation device on the cuff of a smart sock;

FIG. 15 is an exploded view of the stimulation device of FIG. 14;

FIG. 16 is a cross sectional view of the stimulator arranged on an insole;

FIG. 17 is a cross sectional view of the stimulation device of FIG. 14;

FIG. 18 is a perspective view of a smart sock embodiment of a stimulation device including under arch stimulation;

FIG. 19 is a side-bottom view of the smart sock embodiment of FIG. 18;

FIG. 20 is a bottom view of the smart sock embodiment of FIG. 18;

FIG. 21 is a bottom view of an insole embodiment of a stimulation device;

FIG. 22 is a duty cycle diagram showing the adjustment of target cadence;

FIG. 23 is a duty cycle diagram showing an example stimulation pattern; and

FIG. 24 is a perspective view of a stimulation device attached to an adjustable band.

### DETAILED DESCRIPTION

Referring to the Figures, there is provided a preferred embodiment of a stimulation device 1 for guiding movement of a part of a body, preferably a limb. Device 1 includes a plurality of body fixed stimulators 2, configured to provide coordinated stimulus to the body 4, and a control unit 6 in communication with each of the body-fixed stimulators 2. The control unit is configured to generate a control signal 8 to control the body-fixed stimulators 2. The body fixed stimulators 2 are each disposed in a predefined location 10 on the body 4, forming a network of body-fixed stimulators (collectively represented by reference 12). The predefined locations are selected based on the area of the body 4 to which stimulation is to be provided.

The plurality of stimulators may include body fixed miniature vibration motors to deliver haptic cues to skin, speakers to deliver auditory cues, augmented reality to deliver visual cues and body fixed electrical stimulators. Stimulators may be located at various locations on the body, including at least one of an ankle, under the arch of a foot, a shank, a knee joint, lower thigh, upper thigh, a hip joint, between the posterior superior iliac spines, sternum, a collar bone, an upper arm, an elbow joint, a lower arm, a wrist or hand. The optimal location of the stimulators may vary according to each person, and an optimisation process may be carried out to determine the optimal location for stimulators. Additionally, the stimulators may be located on the upper back, with haptic feedback used for posture correction, back pain and/or spinal cord injuries.

In some embodiments, vibration stimulators are located touching the skin and close to bony structures such that the vibration energy is most efficiently transmitted through the bony and tendinous structures of the limb. Advantageously, this means that the stimulators may stimulate multiple peripheral sensing receptors to amplify the stimulation effect. In other embodiments, electrical stimulators are located on a muscle belly, for example, the quadriceps or hamstring groups.

These stimulators are configured to provide coordinated stimulation to the peripheral nervous system (described further below). Preferably, these stimulators are low-cost and may be readily available on the market or can be adapted for use in mobile phones or smart watches. However, it will be appreciated that different types of stimulators may be used. Pharmacological enhancement, deep brain stimulation, and electrical or magnetic transcortical stimulation, attentional cues and emotional cues may be used in combination with the device or as part of the system and can be used to stimulate the central nervous system.

Coordinated stimulation for the peripheral nervous system depends on the target movement pattern and includes: (1) Switching all the stimulators on at the same time; (2) Switching different stimulators on and off at different times according to a predetermined pattern (e.g. setting the stimulation duty cycle to match the target stepping cadence); (3) Switching on some but not other stimulators depending on the movement detected (e.g. switching on one or more stimulators attached to a limb to prevent high frequency tremor when tremor is detected in that limb); (4) Varying the intensity of stimulation according to a predetermined pattern or according to sensor input. (5) Changing the timing and intensity of a predetermined stimulation pattern in response to sensor input (e.g. ongoing adaption of the stimulation duty cycle and intensity considering a user's current versus target cadence).

The optimal stimulation duty cycle for a user may be dependent on the ailment, for example, someone with a leg tremor may have a different stimulation pattern to someone with a drop foot. For stimulation of the feet and ankles, some users may use a left right stimulation duty cycle, which alternates between stimulating the left and right side for a target cadence. Alternatively, some users may use a simultaneous stimulation of left and right stimulators at the same time for a target cadence. For automatic cadence adjustments, if they are consistently stepping in front of the stimulation cycle, the sensors can determine the discrepancy between the step and the stimulation, and the control unit can increase the cadence, or decrease the cadence if the user is stepping behind the stimulation cycle.

The stimulation provided may also include haptic cues to a user's skin using haptic motors, including Vibration Motors (VM), delivered through readily available devices (such as, for example, a smart watch) or through adapted devices (for example, smart socks, anklets, bands, and the like) or through smart garments that have one or more stimulators incorporated into them. For example, a plurality of stimulators may be incorporated into a smart garment such as the band of a sock, or an insole of a shoe (see, for example, FIG. 16 and FIG. 21). Further, a plurality of stimulators may be incorporated into a smart garment such as a pair of tights. For example, the stimulators may be sewn into a pocket of the apparel, or attached to the outside.

In particular, the vibration motor may include a coin or button shaped vibration motor. Coin vibration motors are smaller and are useful for areas which would experience pressure on the motor, such as under the arch of a foot, or to avoid bulk, such as on a wrist. Alternatively, the vibration motor may include a cylindrical vibration motor or an enclosed vibration motor. The cylindrical vibration motor is particularly useful as it can achieve radial and axial vibrational movement when the device is affixed against the skin. The vibration motors may operate at a frequency between 60 Hz to 90 Hz. Stimulation may further include auditory cues using earphones or other mobile speakers or stationary speakers. Additionally, or alternatively, electrical stimulation (ES) could be delivered through the skin using Transcutaneous Electrical Nerve Stimulation or Electrical Muscle Stimulation. Advantageously, it has been found that a combination of aural and haptic stimulation is particularly effective in influencing users to follow the rhythm of the stimulation.

The control unit may be in communication with each of the plurality of stimulators. In an alternative embodiment, the device 1 may include a plurality of control units in communication with each of the plurality of stimulators 2, or in communication with multiple stimulators defining a subset of the plurality of stimulators 2. The control unit includes a computer unit 14, in the form of a controller 16. The controller is configured to generate a control signal 8 which is transmitted to the network of stimulators 12 to control the stimulators to provide stimulation. The plurality of stimulators, or a subset of the plurality of stimulators, may be referred to as a network of stimulators or a stimulation network.

The device 1 further includes a plurality of sensors 18, in communication with the control unit 6. Sensors may include, but are not limited to, accelerometers, gyroscopes, barometers, touch screens, audio sensors (including those which respond to voice activation), electrodes for electromyography, electrodes for electroencephalography (EEG) and pressure sensors which can be incorporated in common apparels including, but not limited to, smart textiles/garments, bands (for example ankle bands, arm bands, wrist bands, thorax bands, head bands, and hats) or pendants. The sensors may be incorporated into the textile itself or may be separately attached to garments. For example, the sensors may be sewn into a pocket of the apparel. The plurality of sensors, or a subset of the plurality of sensors, may be referred to as a network of sensors or a sensor network. In some cases, pressure sensors can include quite a lot of noise and results require filtering. Accordingly, gyroscopes provide an advantageous effect as they are better for obtaining accurate timing of movements, as well as being able to detect the rotation of body parts, such as hands or feet, in different directions. Gyroscopes are particularly good at detecting freezing-of-gait and tremors. Accelerometers are similar advantageous for obtaining precise measurements of movement.

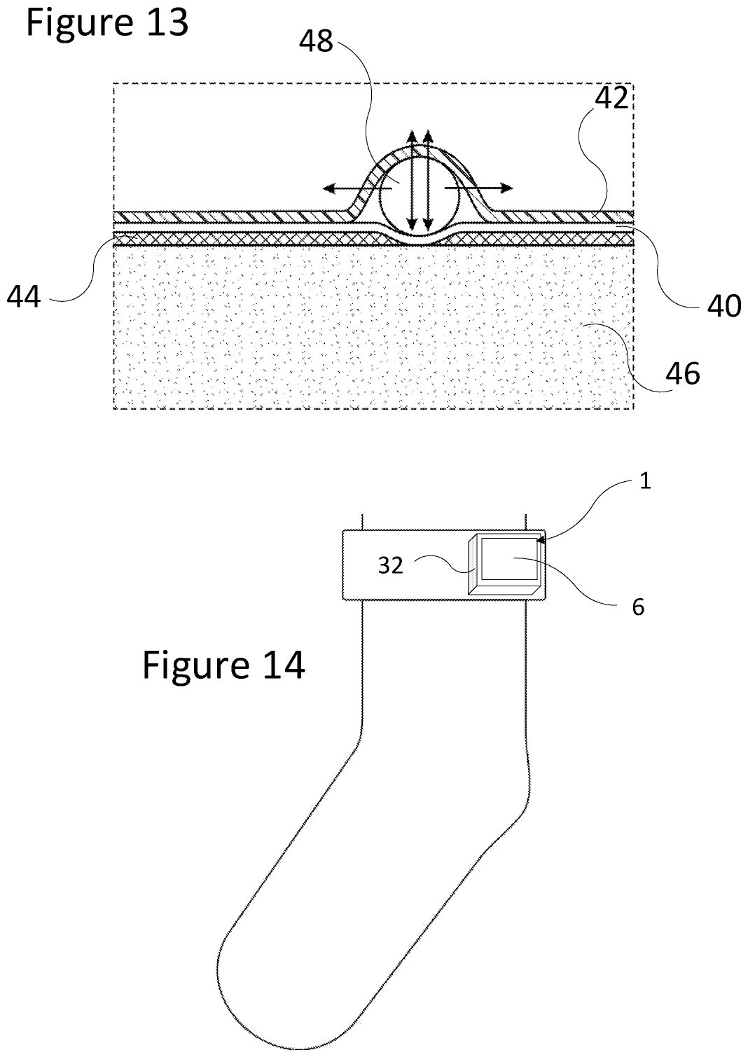

FIG. 13 is a cross sectional view of a stimulator located between two rubber layers (40 and 42), the rubber layers being fixedly attached to the fabric 44 which is worn on the skin 46. The stimulator is a cylindrical vibrational motor, which may move in the directions indicated by the arrows. FIG. 14 is a perspective view of the stimulation device on the cuff of a smart sock. In this embodiment, the stimulator may be located on the ankle, and located directly underneath a casing for the control unit.

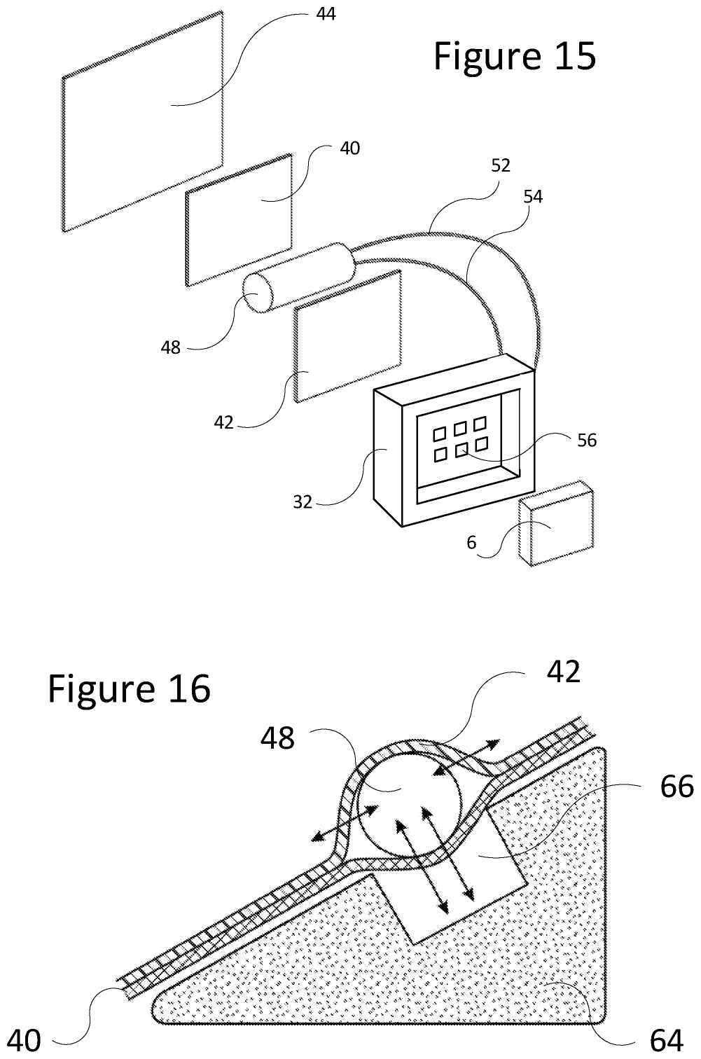

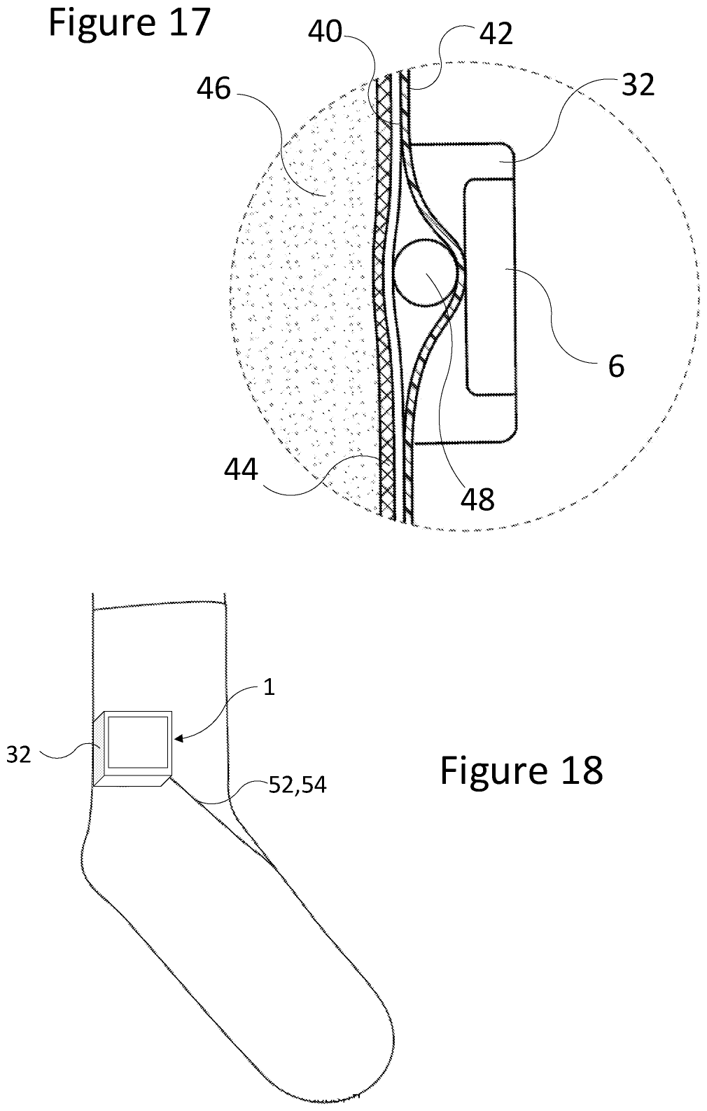

As shown in FIGS. 15 and 17, in one embodiment of the device, a first layer of rubber 40 is fixedly attached to a textile 44, such as a sock or a band. A cylindrical vibration motor 48 is then sandwiched between the first layer of rubber and a second layer of rubber 42, effectively holding the vibration motor in place so as to allow it to move and vibrate effectively, with the right amount of tension. In other embodiments, the device may include a rubber sheath into which the vibration motor is placed, the sheath having at least one open end. A casing 32 is then attached on top of the second rubber layer 42. In some embodiments, the casing acts as a housing for the control unit 6. The second rubber layer may have at least one aperture or slit so as to allow vibration motor leads 52, 54 to protrude through the rubber layer and connect to the casing. The casing 32 may include a plurality of battery connectors 56, to enable a power module to be connected with the casing. In some cases, the casing may form part of the control unit. In other embodiments, the casing may be separate from the control unit, and instead may include an enclosed or unenclosed compartment into which the control unit may be releasably engaged. In some embodiments, the casing may include lowered portions 58 and 60 on its peripheral edge to facilitate removable of a control unit. In some embodiments, the casing may include an engaging mechanism 62, configured to engage with a corresponding engaging mechanism on the control unit.

The control unit may, upon being engaged with the casing, provide power to the vibration motor. The control unit may include a power module. The power module may include a battery. The battery may be a rechargeable battery.

FIG. 16 is a cross sectional view of a stimulator located between two rubber layers (40 and 42), the rubber layers being fixedly attached to a piece of foam 64. In some embodiments the foam may be used as an insole. In some embodiments, the foam may include an indentation 66, configured to enable the stimulator more freedom to move.

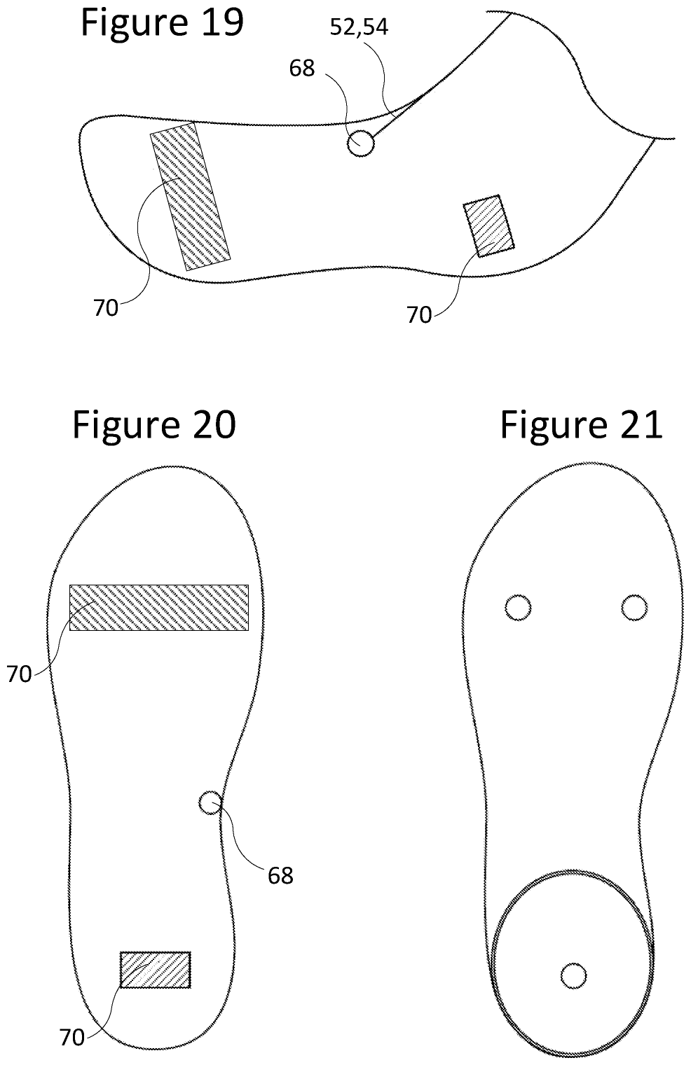

FIG. 18 is a perspective view of the smart sock embodiment, showing the casing 32 attached to the sock. As shown in FIGS. 19 and 20, a stimulator 68 may be fixedly attached to the sock so as to locate it under the arch of the foot. The stimulator is connected to the control module through the casing. In some embodiments, sensors 70 may be located on the balls and heels of the sock. The sensors may be configured to transmit signals to the control unit 6. The sensors may be pressure sensors.

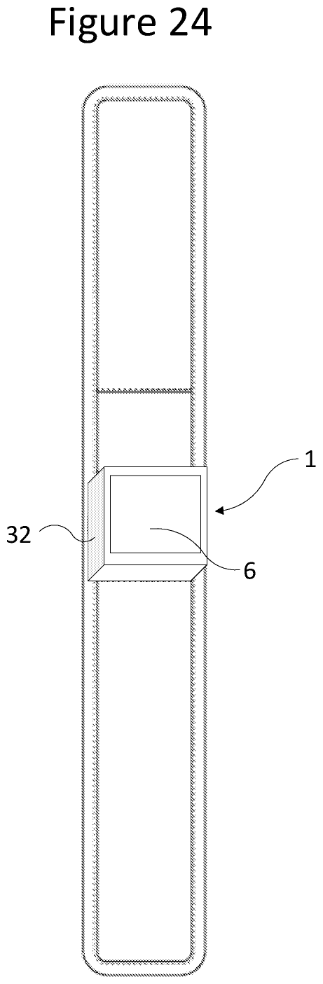

FIG. 24 is a perspective view of the stimulation device attached to an adjustable band 72. The device may be fixedly attached to the adjustable band, and the band may be secured around an ankle or thigh to provide stimulation in a specific area. In this case, the stimulator may be located behind the casing and the control unit. Alternatively, the stimulator may be located at any point along the band and connected to the control unit.

The plurality of sensors is configured to determine the intended target movement being generated by the central nervous system. For example, when the movement being measured is gait, one embodiment requires the user to manually input a direction to initiate the stimulus by pressing a button on a touch screen sensor. Another embodiment uses data from the plurality of sensors and machine learning to automatically determine the intended target movement (including but not limited to walking, reaching, dancing, running, postural transitions, maintaining an upright posture and holding a limb steady to control unwanted movements).

The plurality of sensors is configured to monitor parameters related to the target movement and receive monitoring data 20. For example, when the movement is gait, parameters related to gait movement include at least cadence, step variability, step length, step height, speed thresholds, stimulation intensities, stimulation duty cycles, preferred cadence. However, it will be appreciated that any relevant and appropriate parameters related to gait movement may be monitored.

The control signal optimises and synchronises the two-way information transfer between the central nervous system and peripheral nervous system. The stimulation from the control signal improves sensori-motor integration and may cause new connections to form in the brain, or strengthen existing connections. Additionally, it may reverse symptomatic progression. The control signal 8 is generated when a pre-defined criterion 22 is met. The pre-defined criterion 22 may include a threshold value relating to movement parameters 24. The threshold value may be set manually as a target value 26, or it may be received from historic data 26, monitored data, or data received from a third party. In some cases, the control signal 8 is generated based on at least the monitored data 20. In other cases, the control signal 8 is generated based on historic data and monitored data 20.

Pre-defined criteria may include both manual or automated operation. In the case of manual operation, the user may provide input such as pressing a button or a touch screen, using voice activation or providing audio instruction or activating some other sensing device. In the case of automated or semi-automated operation, the control signal is initiated and/or optimised using data from a plurality of sensors that detect key movement parameters. These include but are not limited to cadence, step length, step-time variability, higher frequency tremor and larger scale unwanted movements. Target movements and unwanted movement variability are determined based on sensor data, user defined settings, machine learning and user input. The pre-defined criteria may be update and/or optimised based on continuous training of the machine learning algorithms employed by the system, as well as pattern recognition algorithms. Users may also provide feedback about their performance with the stimulation after each session, and this can be used to determine if the current control settings may be further improved.

Thresholds for excessive movement variability may be set by the user, a physiotherapist or other trained person or determined automatically by machine learning algorithms that adapt to the user's historic movement patterns.

The timing of the control signal to the stimulation network is fine-tuned using feedback from the sensor network. For example, when the movement is gait, the duty cycle of the control signal will be increased or decreased until it matches the cadence of the user. For example, in the case of a gait training intervention with a prescribed training cadence, the duty cycle is progressively adjusted from the user's current cadence towards the target cadence, while maintaining a good match between the control signal and the user's current cadence, until the target cadence can be achieved.

The intensity of the control signal to the stimulation network may also be adjusted for optimisation. For example, if the user finds the stimulation level too strong or too weak, they may use the control panel to adjust the upper and lower bounds for stimulation. Furthermore, in the case of a gait training intervention, the stimulation intensity is automatically adjusted between the upper and lower bounds based on measured entrainment between the control signal and the target movement. The control panel may be part of the device itself. Stimulation intensity is reduced when the control duty cycle matches the user's current cadence and the pre-defined criteria relating to step variability, step length and step height are met. Stimulation intensity increased when the user's movement does not match the control signal or target movement parameters.

In one example of how the control signal may be optimised, historic data may be utilised to set a range of optimum predetermined criteria. For example, in a case where the movement to be measured is the gait of a user, historic movement data is used to set the cadence within a range of optimum cadences. The control signal may additionally be optimised by encouraging matching of the participant's current or real-time cadence with the stimulation duty cycle. For reducing unwanted or undesirable movements the historic sensor data may be further used with pattern recognition techniques to determine what has historically been unwanted movement. Alternatively or additionally, the historic data may be used to form profiles of desirable or undesirable movement for comparison to real time monitoring data.

Monitoring data relating to the movement of a user from each session is collected by the plurality of sensors and stored, along with the control signals, for the plurality of stimulators. The monitoring data may be stored on the device itself, or on a separate device or database. The monitoring data may also be stored online, such as in a cloud computing environment, or on a third-party server. At the end of a session, users provide feedback about their experience, which is added to stored historic data. Historic data can be used, individually or in combination with the monitoring data received in real time, to train the machine learning algorithms that determine the target movement, automatically adjust the timing of the control signal and automatically adjust the intensity of the control signal.

In another embodiment, there is provided a system 30, including a plurality of body fixed stimulators 2, configured to provide stimulus to the body 4, and a control unit 6 in communication with each of the body-fixed stimulators 2. In the system 30, control unit may be an external computer unit 14, in the form of a smart phone 15a, a tablet, 15b, a smart watch 15c, a wearable device 15d, or a personal computer 15e in communication with device 1 for receiving and, optionally or additionally, combining input data. The control unit may be in wireless communication with the body-fixed stimulators 2. In a further embodiment, the control unit may be connected via a network to the plurality of stimulators and/or the plurality of sensors so as to transmit a control signal. The system may further include a control panel or user interface, configured to receive an input for the control unit. The control panel or user interface may be in direct communication with the plurality of stimulators. Alternatively, the control panel or user interface may be part of a separate program, web application or the like, configured to transmit inputted information to the control unit.

In one embodiment, there is provided a device comprising a plurality of body-fixed stimulators attached to multiple locations and fixed to different body parts. For example, if improved walking is the target movement, stimulators are placed on the lower limbs and/or feet. If improved arm swing is the target movement, then stimulators are laced on the upper limbs, hands, or wrists. By combining stimulation locations, a plurality of target movements can be targeted together. For users who are asymmetric, where one part of their body is worse than the other, the stimulation, or the number of stimulators, can be increased on the most affected side to improve symmetry. The plurality of body-fixed stimulators may be combined with a central control unit and a network of body-fixed sensors. The stimulators, sensors and control unit all being in communication with each other. The device improves the two-way information transfer between the central nervous system and peripheral nervous system. The device helps synchronise the control signals and sensory feedback for the peripheral and central nervous systems. This, in turn, helps to reinforce target neuromuscular signals to improve target movements and dilutes unwanted neuromuscular signals to reduce unwanted movement variability.

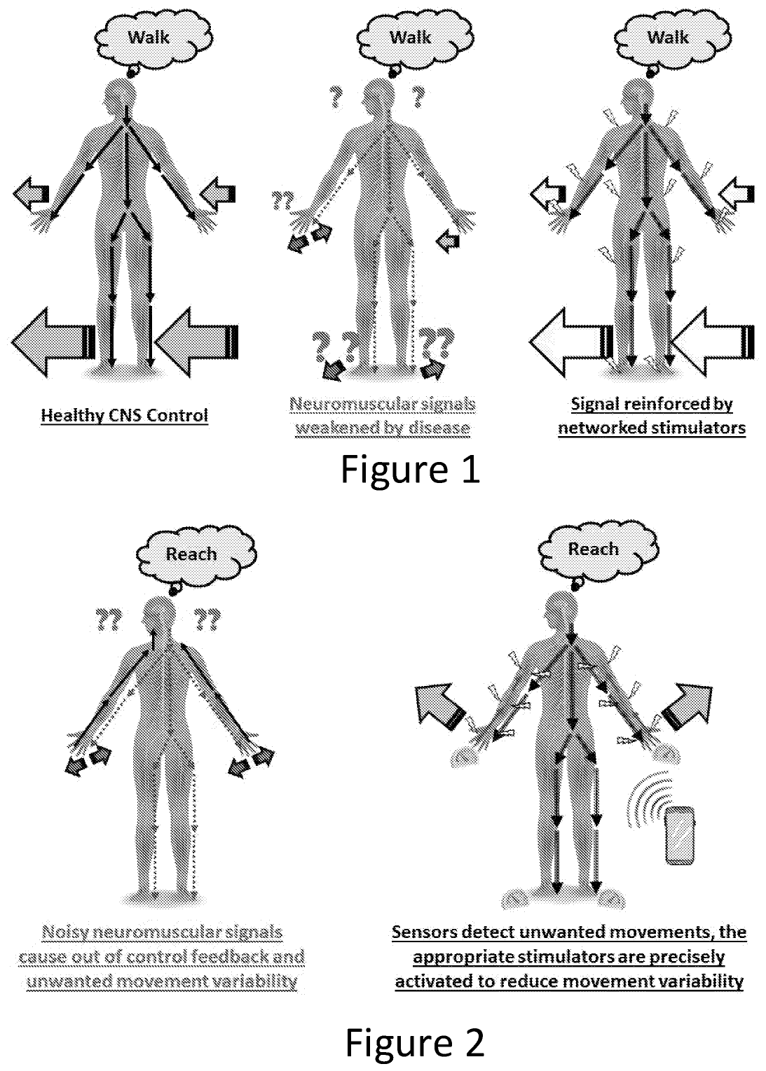

FIG. 1 shows a concept diagram of reinforcing target movements. Control signals exist but are weakened and less coordinated than they should be. However, with appropriately timed and located simulation of the peripheral nervous system, the control signals can be reinforced, target movements improved, and movement variability reduced.

FIG. 2 shows a concept diagram of reducing unwanted movement variability. Neuromuscular signals in the diseased state cause, if unchecked, out-of-control feedback loops to develop and unwanted movement variability. Sensors measure the unwanted movement/neuromuscular signals and activate stimulators in a precisely timed sequence to dilute the unwanted neuromuscular signals and help the user regain control of their movements

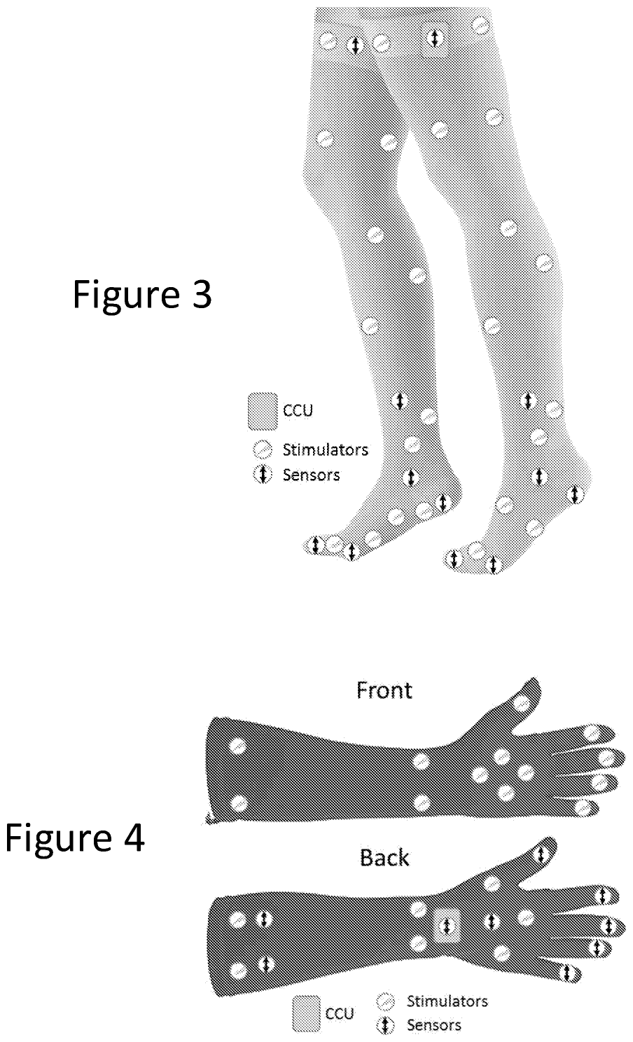

FIG. 3 shows the smart sock embodiment including smart socks, potential locations for the CCU and networked stimulators and sensors on different aspects of the lower limbs. Sensors and stimulators include but are not limited to these locations. It will be appreciated that the preferred location of stimulation depends on the individual and is based on the individual sensitivities of different nerves, the target movement and the underlying cause of unwanted movement.

FIG. 4 shows the smart glove embodiment including smart gloves, CCU on wrist, potential location of networked stimulators and sensors on the front and back of the forearm and hand. Sensors and stimulators include but are not limited to these locations. It will be appreciated that the preferred location of stimulation depends on the individual and is based on the individual sensitivities of different nerves, the target movement and the underlying cause of unwanted movement.

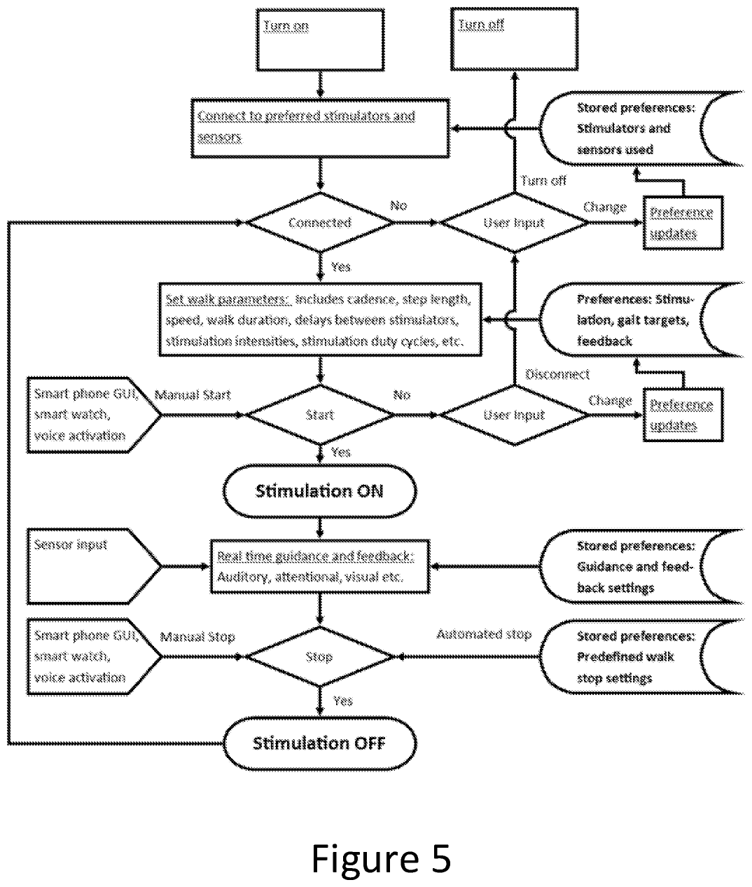

FIG. 5 is a flowchart diagram showing a method of manual operation of the system.

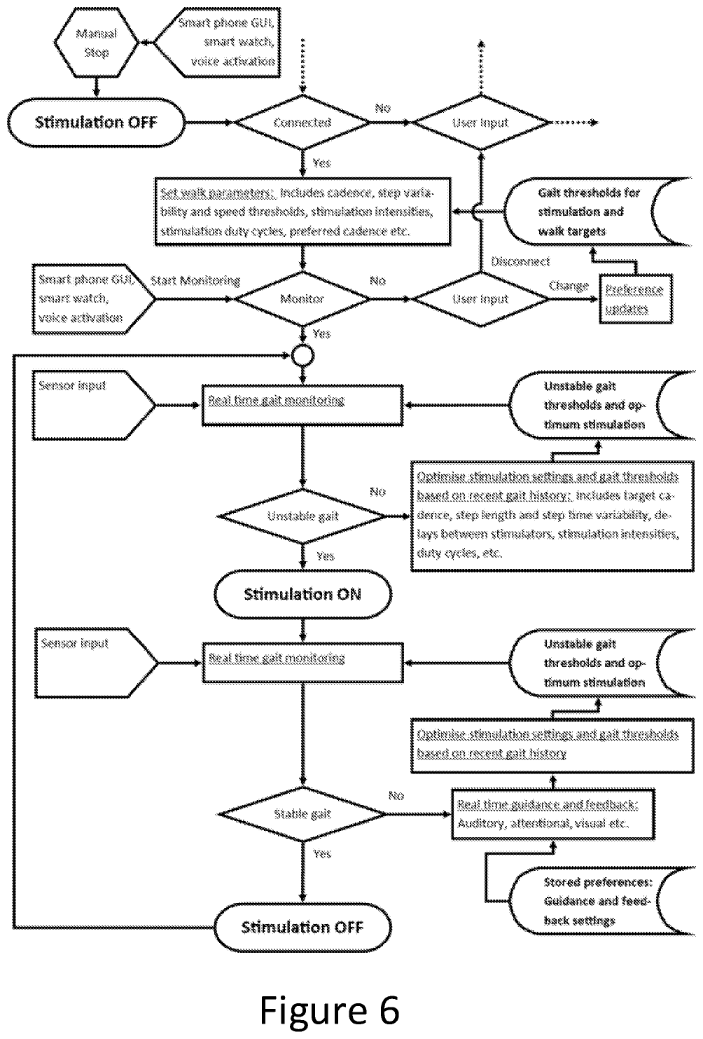

FIG. 6 is a flowchart diagram showing a method of automatic operation (start/stop stimulation) when unstable gait is detected. The real time gait monitoring includes using recent gait history to synchronise the target cadence with the user's current optimum cadence and a machine learning algorithm to detect unstable gait patterns.

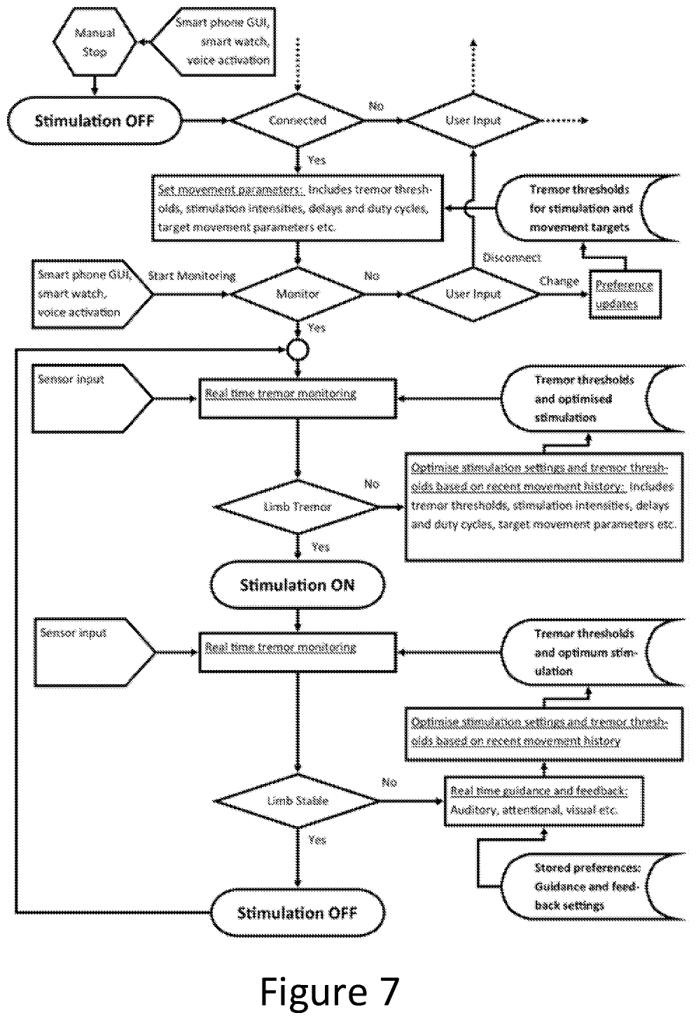

FIG. 7 is a flowchart diagram showing a method of tremor reduction of a limb

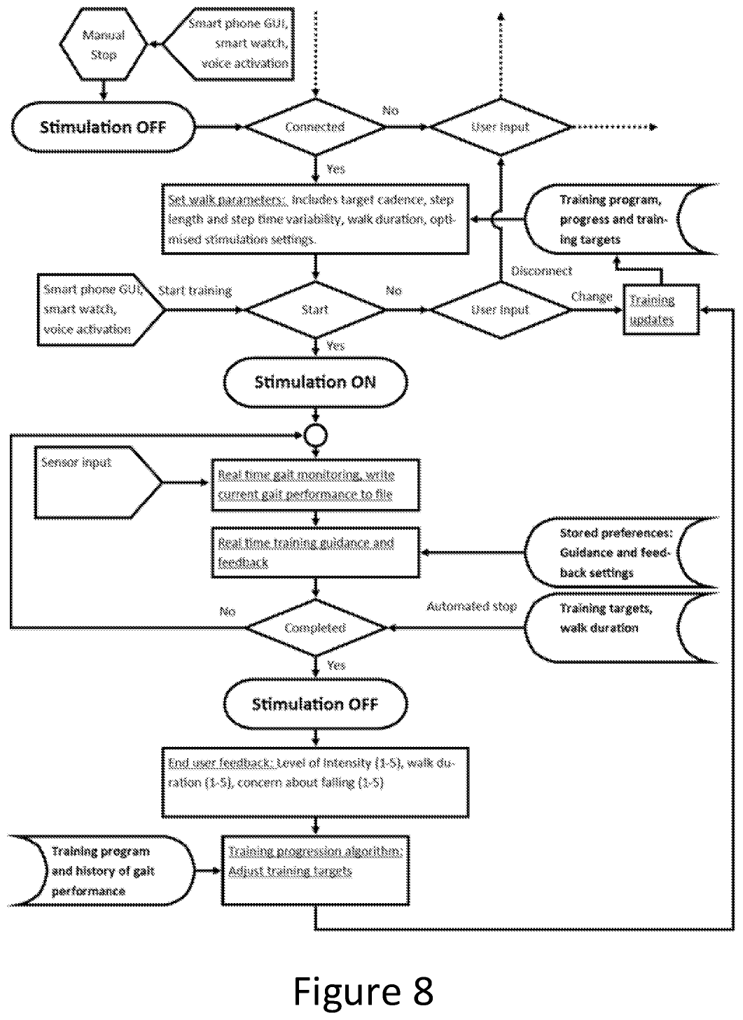

FIG. 8 is a flowchart diagram showing a method of training progression.

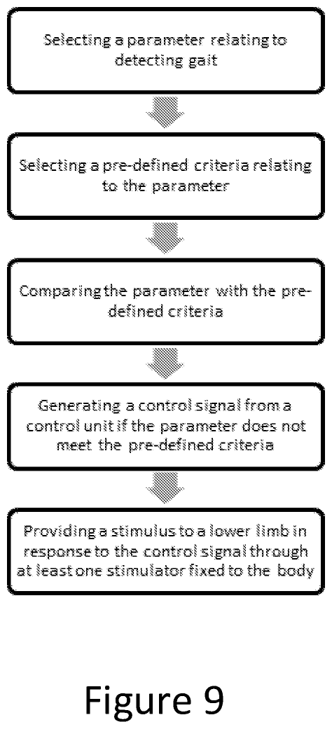

FIG. 9 is a flowchart diagram showing a method according to one embodiment of the invention

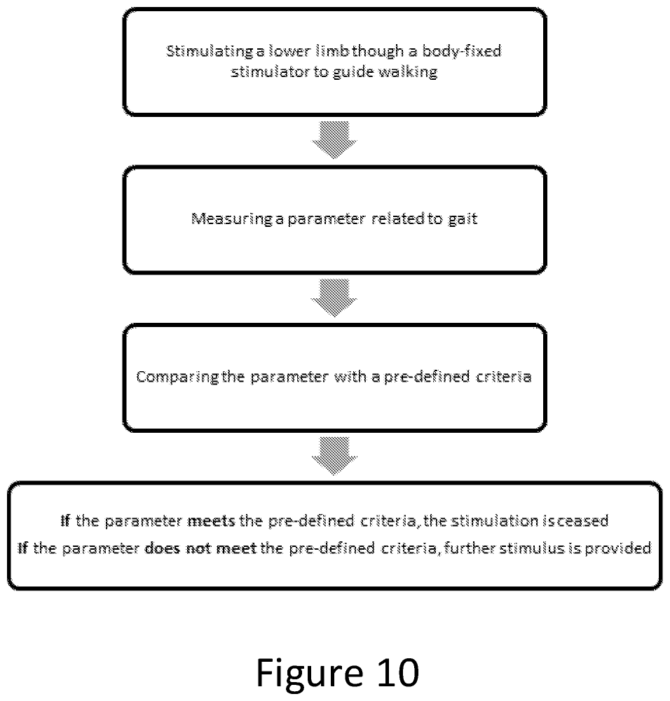

FIG. 10 is a flowchart diagram showing a method according to another embodiment of the invention.

The device or the system may use precisely timed and precisely located low-level stimuli that reinforce existing target neuromuscular signals and dilute unwanted neuromuscular signals.

The device or the system may use coordinated low-level stimuli that reinforce target neuromuscular signals guiding muscle contractions resulting in target movements that are better coordinated within and between limbs and repetitions for the user. For example, there may be noise in the diseased sensori-motor, peripheral and central nervous systems. This may cause unwanted movements and imaginary sensations experienced by the user. The diseased system then tries to compensate for this in a feedback loop, but it is too slow and often gets it wrong—like the positive feedback you get with a microphone speaker plying in to a microphone. This causes more unwanted movements and movement control is ultimately lost.

By providing real coordinated stimulus control signals that link multiple parts of the body in a predictable duty cycle that is matched with a target movement, it is possible to overcome the noise in the system. This then entrains the target movement signals. They piggyback on the real signal and this clears the noise out, providing better overall two-way communication between the central and peripheral nervous systems. In another embodiment, the device or the system may use coordinated low-level stimuli that dilute unwanted neuromuscular signals in the peripheral nervous system and enables improved feedback about the target movement to the central nervous system. This improved feedback guides the central nervous system to generate better control signals, which reduces the cognitive load required by the user to achieve the target movement. For example, for a user who experiences tremor, mechanoreceptors are sensitive to vibratory stimuli. By providing the stimulation, the vibration engages Pacinian corpuscles and modulates the unwanted neuromuscular signals in tremor pathways, thereby, reducing the tremor.

The device and/or system may be considered an exercise aid in which its intended use is to enable a user to better control and coordinate their existing or desired movements.

Desirable movements may include but are not limited to walking, running, dancing, standing upright, maintaining postural balance, undertaking postural transitions, reaching, coordinated hand and arm movements and generally moving in a relatively improved manner. Undesirable movements are generally higher frequency oscillations as detected by Fast Fourier Analysis (for tremor), larger involuntary movements as detected by pattern recognition tools (for dyskinesia), excessive postural sway as detected by wearable devices (for gait, standing and postural transitions), excessive step time variability (for gait), low step height and excessively high cadence (for gait festination).

In particular, the device and/or the system simultaneously guides users to improve target movements through within limb and between limb coordination and may simultaneously or alternatively reduce undesirable movements (such as, but no limited to, limb tremor).

The plurality of stimulators are optimally located at specific positions on the user's body to improve neuromuscular control. Stimulators may be located on the lower limbs, such as on the foot (including under the arch or on the sole, or on the ankle), on the knee, and/or the lower thigh, upper thigh, hip and/or between the posterior superior iliac spines, where leg-related movement such as walking or lower limb tremor is to be improved. Alternatively, or additionally stimulators may be located on the arms, the hand (including on the top of the hand or the palm of the hand), on the wrists, forearms, shoulders, collar bones, sternum or elbow of a user. Stimulators may also be placed on the upper back or neck for posture or spinal-related movements and may even be combined with the sensors to detect if people aren't sitting or standing upright and correct accordingly. Alternatively, a plurality of stimulators may be used for noise cancellation to reduce essential tremor on the hands, legs, and/or head. Additionally, stimulators may be placed in locations where users are experiencing pain so as to distract from the pain. This may be further improved through additional embodiments relating to training and maintenance phases.

The plurality of stimulators and the plurality of sensors are preferably incorporated into smart textiles. The smart textiles allow the stimulators and sensors to be held in place without the need for glue or tape. Smart textiles and/or garments may include smart socks, smart gloves and other wearable garments and textiles.

A key component of providing effective stimulation is the method of attaching the stimulator to the part of the body to stimulate, which depends on the stimulation type. The stimulator should be fixed with sufficient force to be in contact with a body part.

In the case of electrical stimulation, the electrodes need to make contact with the skin and be held in place tightly, which can be achieved through a tight fitting garment, shoe, insole, tape or other adhesive method.

In the case of a miniature vibration motor, the stimulator can be in direct contact with the skin or there can be a layer of material such as part of a tight fitting garment between the stimulator and the skin. A key component of this invention is applying just the right amount of pressure to hold the vibration motor in place. Applying more pressure than required to keep the vibration motor in place may dampen the amplitude of the vibrations and therefore the effectiveness of the stimulation. The optimum amount of pressure required depends on the participant sensitivity, the location of the stimulator and the target movements to be achieved. Optimum contact pressure can be achieved by using an appropriately sized stretchable garment, adjustable bands, other methods of fixing and adjusting the precise location of the stimulator based on the shape of the limb to be stimulated.

Multiple stimulators and sensors may be fixed to a user and connected by wires or wirelessly to the same or different control units. One control unit may connect to multiple stimulators (and sensors). The individual control units are connected by wires or wirelessly either to each other or to a central control unit, which could be an external computer, a mobile phone carried by the user or other mobile device carried by the person. Or in the case of a single control unit, this control unit could be designated the central control unit. The central control unit can also receive inputs from a network of body fixed sensors, which may include but are not limited to inertial measurement units, GPS, pressure sensitive pads, push buttons, switches, keyboard, touchscreen or microphone. The central control unit stores the user's movement history, personal preferences, target movements and information about unwanted movements.

One of the primary mechanisms of the system is that it does not require excessive conscious involvement, such as following a metronome beat (although this may be used as a supplementary stimulus during the training phase). Instead, the system is designed to improve neuromuscular coordination and two-way information flow between the peripheral nervous system and central nervous system at a more automated or lower level of control. In contrast to some devices designed for stroke rehabilitation, it does not provide a mechanical aid (it is not a rehabilitation robot or exoskeleton). The device does not use painful stimuli, invoke pain reflex responses or use electrical stimuli of sufficient magnitude to cause significant unaided muscle contractions. For example, the device is not intended to enable a stroke survivor to grasp and release an object with their hand using electrical stimulation where insufficient capacity exists to achieve at least part of this movement without an aid. Furthermore, the magnitude of the stimulation used in the present invention is not sufficient to cause involuntary movements. The use of augmented reality in combination with the smart textiles/garments is an effective way to reduce and/or prevent freezing of gait.

In one embodiment, the central control unit determines the target movement, the target speed of the movement, the target magnitude of the movement and in the case of cyclical movement like gait the target between cycle variability. The body-fixed stimulators are used to stimulate the parts of the body most appropriate to facilitating the target movement. The stimuli at different locations on the body most appropriate to the target movement are synchronised with the target movement, with varying simulation intensities and time delays, as optimised for the precise parameters of the target movement. The stimulators reinforce the target neuromuscular signals and help to coordinate the user's neuromuscular responses during the target movement.

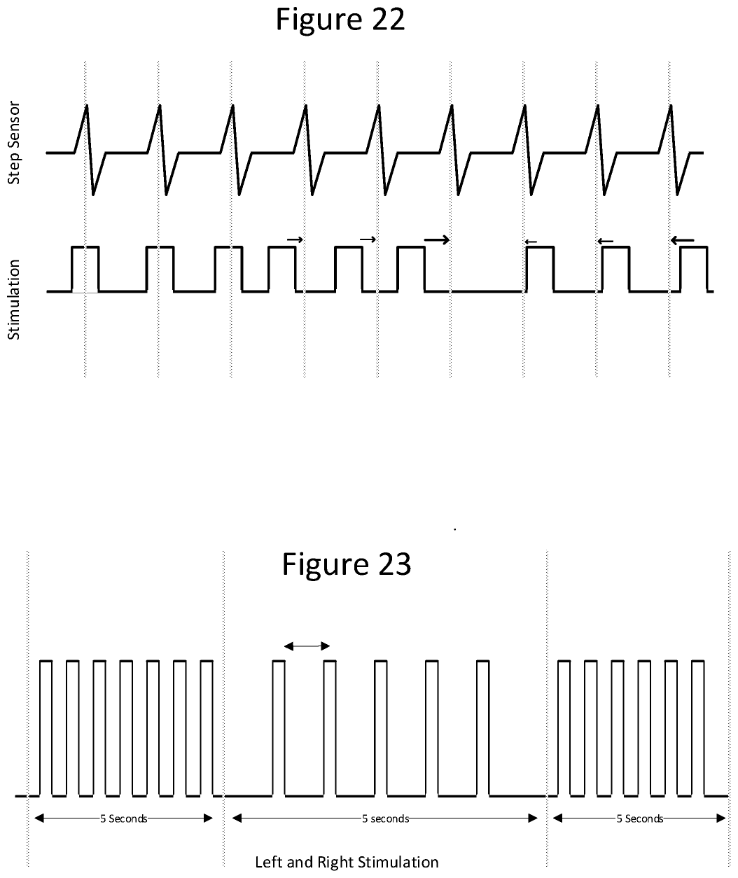

A key factor to the invention is the precise location, optimum synchronisation, timing and intensity of the multiple coordinated reinforcing stimulators and how they adapt to cyclic movements (such as gait) to reduce movement variability. For example, on starting a stimulation cycle, stimulation may be preselected to use a specific stimulation duty cycle (such as left/right alternative or left/right together for foot stimulation). Further, a target cadence may be preselected at the start of the movement cycle, or a target cadence lower than the expected cadence may be selected, and the target cadence is then increased to an optimum level based on the feedback and measurement data being received by the sensors. Further, on starting a stimulation cycle, the sensors may determine that the user is performing a target movement faster than expected or slower than expected, in which case the optimum movement timing can be adjusted accordingly. For example, see FIG. 22, which shows the duty cycle of the adjustment of stimulation to match the cadence.

In some embodiments, sensor feedback is not essential. The device may coordinate stimulation to multiple locations on the body based on a prescribed and progressive training program. Feedforward control is used to achieve a desired training outcome. For example, a walk program comprises the below progression. Visual cues or indicators and a rhythmic audio cue such as a metronome beat matching the stimulation duty cycle may be used. As a user progresses, the walking periods may be increased, and the rest period reduced in addition to the preferred cadence being increased.

i) 30 seconds warm up walking—stimulation duty cycle at preferred cadence.

ii) 2 minutes walking preferred speed—stimulation duty cycle at preferred cadence.

iii) 30 seconds rest—no stimulation

iv) 2 minutes slow walking—stimulation duty cycle 20% below preferred cadence.

v) 30 seconds rest—no stimulation

vi) 2 minutes walking preferred—stimulation duty cycle set at preferred cadence.

vii) 30 seconds rest—no stimulation

viii) 2 minutes walking fast—stimulation duty cycle at 20% above preferred cadence.

ix) 30 seconds rest—no stimulation

x) 2 minutes walking preferred—stimulation duty cycle set at preferred cadence.

xi) 30 seconds Warm down—stimulation duty cycle at preferred cadence.

In another embodiment, the body-fixed sensors detect unwanted movements (including tremor, excessive postural sway, freezing of gait or excessive movement variability). For example, in one embodiment, where the movement being measured is gait, the regular gait cycle consists of stance phase (where the foot is relatively stationary) and swing phase (where the foot is moving) with a dominant frequency proportional to the stepping cadence. Unwanted movements are higher frequency movements not at a harmonic of the dominant stepping frequency as determined by fast Fourier transform or other appropriate method. Pressure sensors disposed in a smart sock or a shoe obtain monitoring data in the form of pressure measurements to detect when the foot of a user is in contact with the ground. This monitoring data is combined with monitoring data obtained from gyroscopes and accelerometers to detect lower limb tremor. The lower limb tremor is determined by disturbance to the gait stance/swing cycle and/or the presence of high frequency oscillations that are not at a harmonic of the dominant stepping frequency and machine learning. For example, in the case of upper limb tremor or unwanted upper limb movements, a machine learning algorithm is trained to detect tremor and unwanted movements using monitoring data and/or historic data from the body-fixed sensors. During training of the machine learning algorithm, the user may be required to undertake a number of activities of daily living, with and without tremor and unwanted movements as determined by the participant and/or a trained person. Data from these movements are recorded with the body-fixed sensors and stored as part of the user's historic movement data.

Excessive postural sway may be detected by digital inclinometers and other sensors attached to the user's pelvis, torso or other body parts and machine learning algorithms that adapt to the user's historic movement patterns and takes into account individualised thresholds that may or may not be set by a physiotherapist or other trained person.

Voluntary stopping can be distinguished from involuntary stopping by the absence of unwanted high frequency movements, excessive postural sway, through manual input (e.g. pressing a button, inputting information through a touch screen, audio instruction, or the like) by the user and machine learning algorithms that adapt to the user's historic movement patterns and takes into account individualised criteria or thresholds that may or may not be set by a physiotherapist or other trained person.

Excessive movement variability is determined by data collected from the sensing network. This may include but is not limited to variability of timing between cycles, variability of movement amplitude and variability of cyclic trajectories. Thresholds for excessive movement variability may be set by the user, a physiotherapist or other trained person or determined automatically by machine learning algorithms that adapt to the user's historic movement patterns.

The central control unit automatically determines which stimulators need to be activated to enable the user to better control the unwanted movements. The central control unit uses machine learning algorithms based on data from the plurality of sensors and the device configuration to determine which stimulators to activate. In addition to the automated detection, the user can also activate (or deactivate) the central control unit or individual stimulators manually at any time. The stimulators at different locations on the body are activated asynchronously or synchronously to control the unwanted movements.

The stimulators to be activated may be initially determined by a default configuration. The default configuration can be updated by the user with or without the assistance of a trained person through a calibration procedure. The calibration procedure requires to user to undertake tasks (such extending the arm with or without holding a weight in the hand for an extended duration or turning on the spot) that can cause unwanted movements. The default configuration settings and/or locations of the stimulators may then be adjusted to optimise the reduction of unwanted movement.

The stimulators reinforce target movements and/or dilute the unwanted neuromuscular signals and help to the user to regain control of their neuromuscular responses. A key factor to successfully improve target movements and/or reduce unwanted movements is the precise location, optimised timing and the intensity of these multiple diluting stimulators and how they adapt to cyclic movements (such as gait) to reduce movement variability. The precise location of each stimulator is optimised by feedback from the user. The default locations are adjusted during a calibration procedure until the perceived stimulation intensity (as perceived by the user and/or measured by a trained person) is optimised.

For example, real-time feedback is provided by the sensors (e.g. pressure sensors in contact with the soles of the feet) that detect stepping events. Steps are used to calculate the instantaneous step-time variability (from the last eight steps). Stimulation is triggered by either excessive step-time-variability or freezing of gait. For example, when excessive step-time variability is detected, rhythmic stimuli are provided at a user's preferred cadence; when freezing of gait is detected, a continuous haptic stimulus is delivered or pulsed stimuli at a cadence lower than the user's preferred cadence.

In another embodiment, the device uses synchronised stimuli (haptic, visual and auditory) to retrain the neuromuscular system to walk better. After completing training, a minimal set of stimulators controlled by a mobile device (for example, a smart phone or a smart watch) is used for ongoing benefit. For example, initially a user may require an intense training period with multiple stimulators attached to each leg used in combination with a gait re-training program to make significant improvements. After learning the new movement, less stimulation may be required. Training may be completed with stimulation in combination with auditory and visual cues. It may be sufficient to use one stimulator for each foot (attached either to the foot arch or to the fibula just above the lateral malleolus of the ankle) without additional training aids. The user can then use this minimal set of stimulators at their discretion (controlled by their preferred mobile device, smart watch or other personal device) to improve their mobility and increase their physical activity level during daily life. Improved mobility and increased physical activity level are linked to multiple health benefits.

The further embodiment uses the network of synchronised stimulators to reduce unwanted variability while performing arm and hand movement tasks. Other embodiments exist, which are not described in detail in this document.

These other embodiments include but are not limited to assisted body transfer training (from sit to stand, from lying to standing, and from standing to walking), voluntary step training, reactive balance recovery training, strength or balance training, dancing, running, sports and athletic performance. For example, a duty cycle which may be used for balance training is shown in FIG. 23.

In another embodiment, there is provided a method of providing stimulus to guide target movements combined with a progressive training program directed at improving the user's ability to perform the target movements.

Advantageously, stimulation may result in long term improvement in target movement or reduction in unwanted movement, which persists after the stimulation has ceased.

In a further embodiment, there is a system including (i) at least one of a stimulation suit, multiple stimulation garments or stimulation devices; (ii) a sensing system configured to analyse the target movement, and (iii) an analysis and control system. In some embodiments, the user is instructed to perform repetitions of the same target movement (such as walking). With each repetition, different stimulators may be used with different settings and the target movement analysed. Through a process of search optimisation, the user's optimum number and type of stimulators, locations of stimulation, intensity, frequency and duty cycle of stimulation are determined.

In this document. “target movements” include, but are not limited to, walking, running, dancing, postural transfers, maintaining an upright posture, control of posture while moving, arm swinging, hand reaching towards an object, picking up an object, carrying the object, directed movement holding the object and putting the object down.

In this document, “improve” includes, but is not limited to, guiding movement, improving movement intensity, improving movement timing, improving movement accuracy, improving movement precision, improving responses to postural perturbation and improving postural balance.

In this document, “unwanted movements” or “undesirable movements” include, but are not limited to, movements that are not the target movement and which would detract from the target movement if not controlled.

In this document, “reducing” includes, but is not limited to, reducing movement variability, reducing movement fatigue, reducing postural tremor, reducing postural sway, reducing hand tremor, reducing limb tremor and reducing joint tremor.

### Embodiment—Smart Socks

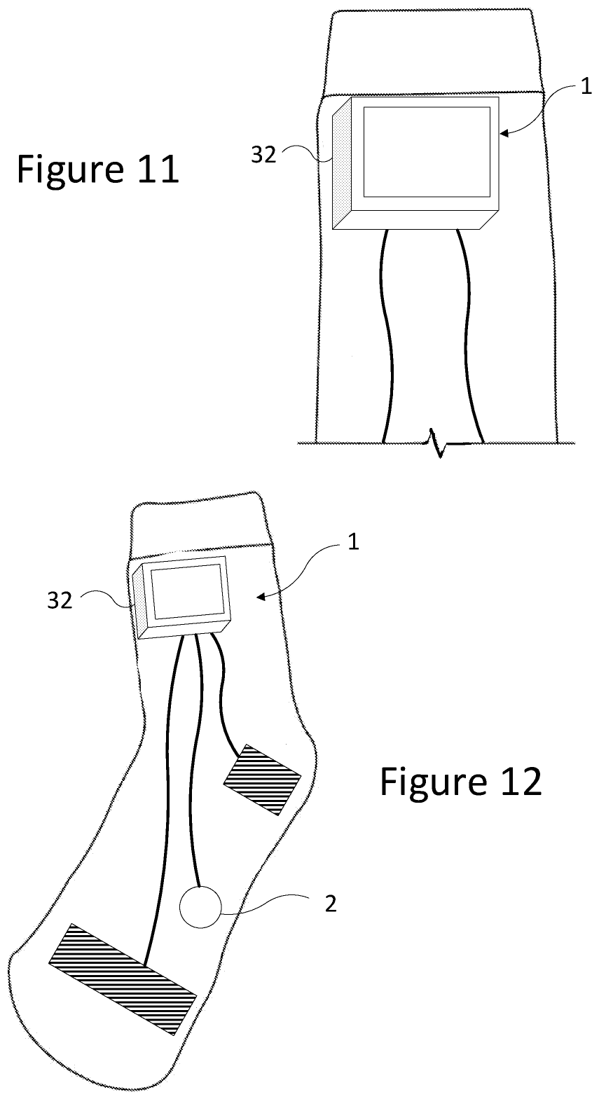

FIG. 11 shows an enlarged perspective view of a smart sock embodiment, showing a casing 32 of the control unit 6. FIG. 12 is a perspective view of the smart sock embodiment, showing stimulators fixedly arranged on the sock, and connected to the casing of the control unit.

In this embodiment, a user wears a pair of smart socks which may be a part of a larger stimulator and sensor network. The smart socks contain several sensors and stimulators and are connected to the user's mobile phone, smart watch, personal computer or wearable controller, which can be the central control unit. As shown in FIGS. 11 and 12, a key component of this embodiment is the location of the primary stimulators either under the arches of both of the user's feet and/or at the ankle positioned on the fibula just above the lateral malleolus depending on user preferences. The stimulators used are small coin vibration motors or other small vibration motor. Alternatively, the stimulators used may be cylindrical vibration motors. At the foot arch, these stimulators primarily excite cutaneous nerves at the foot, which are not usually in contact with the ground during walking. At the fibula, these stimulators primarily excite cutaneous nerves at the ankle. These stimulators also transmit vibrations more through the bony structures and connective tissues of the lower limb that subsequently excite more widely the nerves of the lower limb. Additional stimulators may be placed using leggings, knee high socks or tight-fitting shorts or leggings on the shank or thigh of the leg or pelvis to stimulate nerves in the skin, tendons connective tissue or muscle fibres of the lower limb. As shown in FIGS. 18 to 20, the sensing network may comprise pressure sensors in the sole of the sock and inertial sensors, EMG heart rate or skin temperature sensors at the shank or ankle. However, it will be appreciated that any appropriate stimulators and sensors may be used and may be placed in any appropriate location.

By detecting and reacting to the user's movements, the smart socks assist a user to walk, run, dance and move in a relatively improved manner. Specifically, the use of synchronised networked stimulators underneath the arches of both feet to prevent freezing of gait is particularly advantageous. This embodiment includes a number of sensors and stimulators that are connected to the user's preferred mobile device (including but not limited to mobile phone, smart watch, personal computer or wearable controller), which can be the central control unit. The central control unit generates a control signal that precisely coordinates these networked stimulators to achieve the target movement. Data from the sensing network and machine learning are used to determine the target movement parameters such as cadence and step-time variability and fine-tune the control signal to optimise gait performance. However, it will be appreciated that the smart socks are not limited to preventing freezing of gait and are not limited to use in assisting users with specific ailments, such as Parkinson's disease, but rather may also be suitable as a general exercise aid. Additional details of how movements can be trained and/or guided using the smart socks are provided in further embodiments.

### Embodiment—Smart Gloves

In a second embodiment, a user wears a pair of smart gloves or a single glove, which may be part of a larger stimulator and sensor network. The smart gloves contain a number of simulators and sensors and are connected to the user's preferred mobile device (including but not limited to mobile phone, smart watch, personal computer or wearable controller), which can be the central control unit. The stimulators are incorporated into smart gloves and may be located both on the anterior and posterior sides of the user's hands and forearms. Sensors on the smart glove detect hand and arm movements. The primary sensors include accelerometers and gyroscopes, which detect translational and rotational movements respectively and pressure sensors that detect when the participant is holding an object. The central control unit (CCU) determines which movements are target movements and which movements are undesirable movements. Target movements are determined based on sensor data, user defined settings, machine learning, user input and historic movements using pattern recognition techniques. Undesirable movements are also determined based on sensor data, user defined settings, machine learning and user input. Undesirable movements are generally higher frequency oscillations as detected by Fast Fourier Analysis (for tremor) or larger involuntary movements as detected by pattern recognition tools (for dyskinesia). Stimulation is then used to assist the user to achieve relatively improved control and coordination of their hand and arm movements. On anterior side of the upper limb, stimulation may be applied to the fingertips, palm, wrist and to the proximal forearm. On the posterior side of the upper limb, stimulation may be applied to the metacarpal, carpus, ulna, radius and to the proximal forearm. At the fingertips and other soft tissue locations, these stimulators primarily excite localised cutaneous nerves. When located on bony structures, these stimulators also transmit vibrations more widely through the bony structures of the upper limb that subsequently excite more widely the nerves of the upper limb. It will be appreciated that the preferred location of stimulation depends on the user and is based on the individual sensitivities of different nerves and the underlying cause of limb tremor. The coordinated stimulation of the peripheral nervous system provided by the stimulators overcomes sensory noise and dysfunctional feedback control loops. The control signal for the stimulus is matched with the intended target movement, which improves the synchronisation of information flow between central nervous system and peripheral nervous system. Improved information flow further also assists the central nervous system to generate more accurate control signals.

A key component of this embodiment is the location of sensors and stimulators on the hand and forearm, the timing and intensity of stimulation and their incorporation into a pair of gloves. The CCU may be worn at the wrist and could also be a smart watch or similar device.

### Embodiment—Walking Performance with Smart Garments

### Training Phase

In a third embodiment of the invention, the training phase may include, but is not limited to, one or more of the following components: training of upright posture, training of arm swings, training of leg movements, and training of different aspects together. The invention then transfers to the maintenance phase.

A network of body-fixed stimulators (and sensors) are woven into a tightly fitting garment and worn by the user. For visual stimulation, one or more screens (e.g. tablet computer, laptop, desktop computer, tv, smart phone, virtual reality device, augmented reality device) displays the target movements (or in absence of a screen a clinician may act out the role of a virtual trainer). Auditory stimulation is provided by wireless headphones, speakers or in the absence of this technology, a clinician. Electrical stimulation is provided through electrodes in contact with the skin. Vibration stimuli are provided by miniature vibration motors held next to the body. Training is driven through a central computer with wireless or wired connections to all stimulation and sensing components. Training could be clinic-based, centre-based or home-based.

During the upright posture training, two or more VM and or ES will be applied on the upper back, between the shoulder blades, and or lower and middle back. The stimuli are used to remind the user and/or stimulate the muscles to align back, neck and shoulder. The sensor network will determine if the user is slouching or moving into a stooped posture and will cause the stimuli to fire appropriately. Sensors may be placed on the user's torso to sense the stooped posture more sensitively. The simulation and sensor networks are woven into a tight fitted garment.

During training, the user stands in front of a screen following the instructions of the virtual trainer. The trainer provides a visual representation of the ideal target movement. The trainer maintains correct posture and the training upper body movements are synchronised with the body-fixed stimulators to facilitate control of posture during both trunk extension, trunk flexion and trunk abduction. Alternatively, instruction and demonstration can be given by clinician or video clip. Auditory cues and encouragement are provided by headphones or speakers synchronised with the body fixed stimulators. The body fixed sensing network provides feedback on the correct postural alignment, accuracy, magnitude and timing of trunk movements. Feedback to the user is provided by visual, auditory or haptic methods. The correct attainment of target posture and target movement intensity is required for progression from slow simple movement patterns to faster more complex sequences of movements.

Once upright posture training is successfully completed, training may progress to arm and then leg movements. During the subsequent training of arm and then leg movements the user is reminded by the stimulators attached to the torso to maintain proper postural alignment. This may cause new neuromuscular pathways to be formed and as training progresses to the maintenance phase. As training progresses, proper postural alignment becomes a more automated neuromuscular function with minimal attentional involvement required.

During arm swing training, which trains arm swing with a focus on swing magnitude, timing and control of swing variability, the focus is on using the stimulators and sensors fixed to the user's torso, arms, hands and/or wrists. The user stands in front of a virtual trainer. The virtual trainer's arm swings are synchronised with the body-fixed stimulators to facilitate arm swings with the target magnitude and timing. Auditory cues are provided by headphones or speakers such as “left”, “right”, “stop” and are synchronised with the body fixed stimulators.

For example, the user might stand with arms hanging in a neutral position at each side of the waist. For example, training may start with single arms swings at a slow speed (3000 ms per cycle). Example stimulation schedule includes: The right arm bicep VM fires synchronously with the left arm triceps VM both for 250 ms to initiate the movement. Synchronously a “right” auditory cue is given and visual stimulation by the virtual trainer moving the right arm forward and left arm back.

At 1000 ms the wrist VMs on both sides fire for 100 ms marking the forward most position of the right arm and rear most position of the left arm. Synchronously a left right arm bicep VM fires and the right arm triceps VM fires both for 250 ms. Synchronously a “left” auditory cue is given and visual stimulation by the avatar starting to move the left arm forward and right arm back. At 2000 ms the wrist VMs on both sides fire for 100 ms marking the forward most position of the left arm and rear most position of the right arm. Synchronously a “stop” auditory cue is given and visual stimulation provided by the virtual training starting to return the arms to a neutral position. At 3000 ms the wrist VMs on both sides fire for 100 ms marking a return to a neutral position for both arms.

Sensors attached to the user's wrists provide feedback on the timing and magnitude of movement. Feedback is displayed on the screen. When the required number of repetitions is completed at the target magnitude and the target speed, training may progress.

The progression is from single arm swing detailed above at 3000 ms per cycle, to multiple arm swings without a stop at 2000 ms per cycle in the form “left”, “right”, “left”, “right”, “left”, “right”, (which may be repeated for 60 seconds), “stop”, with an extra 1000 ms provided for the “stop” movement at the end of the sequence. Sensors attached to the user's wrist provide feedback on the timing and magnitude of movement. Feedback on attainment of target magnitude and timing is displayed on the screen. When the required number of repetitions has been completed at the target magnitude, the target speed, and below the movement variability threshold, training progresses.

The progression is from multiple arm swings at slow speeds (around 2000 ms per cycle) to multiple arm swings at moderate speeds (around 1500 ms per cycle) to multiple arm swings at fast speeds (around 1000 ms per cycle).

During, walking on the spot training, which includes walking on the spot with good knee height and control of foot trajectory, the focus is on using the stimulators and sensors fixed to the user's torso, legs, ankles and/or arches of the feet. The user stands in front of a screen looking at the virtual trainer. The virtual trainer's leg movements are synchronised with the body-fixed stimulators to facilitate rhythmical stepping with progressively greater knee height. Auditory cues are provided by headphones or speakers “left”, “right”, “stop” synchronised with the body fixed stimulators. Training progresses from simple to complex movements at slow to fast speeds as previously outlined for arm swings.

During combined training of legs, arms and upright posture (walking on the spot), all available stimulators and sensors are used. The user stands in front of a screen virtual trainer. The virtual trainer maintains an upright posture while walking on the spot with progressively greater arm and leg movements. Full body movements are synchronised with the body-fixed stimulators to facilitate rhythmical and coordinated whole body movements. Auditory cues are provided by headphones or speakers “left”, “right”, “stop” synchronised with the body fixed stimulators. Training progresses from simple to complex movements at slow to fast speeds as previously outlined for arm swings and leg movements.

For assisted walk training, all available stimulators and sensors are used. The user may walk on a treadmill with visual cues (or indicators) projected on the treadmill surface or walk across a flat surface. Auditory cues are provided by headphones or speakers “left”, “right”, “stop” synchronised with the body fixed stimulators. In addition, users may be guided by visual cues (stepping targets), augmented reality or guided by a clinician who walks beside them (for safety and to set the pace) or by a virtual trainer on a screen modelling the target walking movements. The user is guided by the progressive training to simultaneously maintain and upright posture, use coordinated arm swings, target step lengths and toe clearance in the swing phase. Training progresses from slow to fast speeds as previously outlined for walking on the spot.

The training phase can be any length of time, which may depend on the user's progress. The training phase may use multiple stimulation modalities including haptic cues as well as auditory cues (e.g. metronome, music etc.) or visual cues (e.g. augmented reality, a virtual trainer, clinician who demonstrates the movements, indicators on the ground, a grid or stepping mat etc.), which do not all have to be body fixed, to retrain existing human movements or train new human movements in a controlled environment. The training phase requires increased attentional focus. The training phase is progressive with respect to movement complexity, movement speed and movement intensity. The training phase requires the user to attempt to complete the target movements as dictated by the various cueing mechanisms or stimulators to strengthen existing neuromuscular, spinal and central nervous system (CNS) pathways and develop new pathways in a controlled environment.

During training, the invention may cause new pathways to be formed in the CNS, at the spinal level and/or neuromuscular responses to be fine-tuned to facilitate better target movements. The training phase requires the user to attempt to control the unwanted movements while being stimulated in a controlled environment. In the training phase, the level of stimulation and target movement parameters (speed, timing, intensity, magnitude) are dictated by the progressive training program. As part of the progression, the level of stimulation will be reduced (if feasible) and the level of difficult of the target movement parameters will be increased. Goals for the training phase may be set by the user, the treating clinician or by a support person.

### Pre-Maintenance Stimulation Optimisation Phase (Gradual Withdrawal of Redundant Stimulus)

At each progression detailed above in the training phase, after initial training for each movement has been completed successfully, training for that movement is repeated with a reduced set of simulators (e.g. wrist only in the case of arm swings) which when activated evoke the newly trained movements. If the user is unable to achieve the target movements with a limited number of stimulators, more stimulators are added. If the user is successful, more stimulation is withdrawn and/or the stimulation magnitude is reduced until the optimum amount of stimulation for the user to achieve the target movements with acceptable performance is determined. This information is used to inform the optimum stimulation level required during the maintenance phase.

The “pre-maintenance phase” can be any length of time. Compared to the training phase, in the pre-maintenance phase the stimulation is progressively withdrawn. In the pre-maintenance phase, the optimal level of reduced level of stimulation to evoke the existing and/or previously trained neuromuscular pathways is determined. In the pre-maintenance phase, the objective is to reduce the attentional demand and move towards automated targeted responses with the optimum subset of stimulation.

### Maintenance Phase

In the pre-maintenance phase, the optimal amount of body-fixed stimulators is established for the user. The optimal amount of stimulation is determined in consultation with the user and based on a trade-off between intervention efficacy, battery life, comfort and usability. Starting with the minimum amount of stimulators (generally one stimulator located on each lower limb at the user's preferred location), additional stimulators are added until the target gait parameters are achieved. Target gait parameters include but are not limited to cadence, step length, step height and step-time variability. These stimulators will remain available to users in daily life for use in a free walking maintenance phase with optimised stimulus, and will be guided by user's preferences and lifestyle. The user selects (with or without the assistance of a trained clinician) using their preferred mobile device when the stimulators will be activated. The user also selects if the simulation starts manually or in response to the automated detection of impaired gait. The optimum network of stimulators and sensors will be woven into smart garments. The user's mobile phone or smart watch may be used as the central control unit.

The “maintenance phase” can be any length of time. Compared to the training phase, the maintenance phase uses a reduced set of body fixed stimulators and sensors and can be undertaken in any environment. In the maintenance phase, the optimal level of stimulation is used to evoke the existing and/or previously trained neuromuscular pathways and does not necessarily require increased attentional focus from the person. In the maintenance phase, the target movements and level of stimulation are set and adjusted according to the user's preferences either by the user, a support person or a health care professional. In the maintenance phase, the target movements and level of stimulation can be automatically adjusted in real-time based on the body-fixed sensor network.

### Embodiment—Reducing Unwanted Movements During Arm and Hand Movement Tasks

In a further embodiment of the invention, the training starts with holding the arms stationary, to moving the arms to and from different stationary positions and progresses to more complex movement tasks with focus on training the precision and speed of target movements, while minimising the unwanted movement variability.

A network of body-fixed stimulators and sensors are woven into a tightly fitting garment and worn by the user. The user will practice the movement with objects, varying in size and weight and varying types of grip handles. Target movements are synchronised with precise stimulation of the torso and arms to reaching to improve hand or arm movement performance.

During training, when the sensor network detects unwanted movements (typically higher frequency oscillations). The central control unit calculates the axes of the unwanted movements and determines the precise timing, period, magnitude and delays for stimulation to optimally reduced the neuromuscular signals causing the unwanted movement variability.

Tremor control training may include throwing and catching balls in each hand and between left and right hands and may progress to juggling multiple balls of different masses. In one embodiment, the vibration stimulators are located close to bony landmarks such as the sternum, clavicles, elbows and wrists. The vibration stimulation duty cycles are used to precisely guide the timing of ball throws and catches. Auditory cues may also be used to guide timing of throws. Simultaneously, electrical stimulators attached to the muscles that actuate the upper limb and are used to cancel out the high frequency tremor through stimulation that precisely opposes the high frequency tremor measured by sensors attached to the upper limb.

One embodiment uses the network of synchronised stimulators to reduce unwanted variability while performing arm and hand movement tasks. Target movements and unwanted movement variability are determined based on sensor data, user defined settings, machine learning and user input. Unwanted movements are generally higher frequency oscillations as detected by Fast Fourier Analysis (for tremor) or larger involuntary movements as detected by pattern recognition tools (for dyskinesia).

These other embodiments include but are not limited to assisted body transfer training (from sit to stand, from lying to standing, and from standing to walking), voluntary step training, reactive balance recovery training, dancing, running, sports and athletic performance.

In a further embodiment, the device uses synchronised stimuli (haptic, visual and auditory) to retrain the neuromuscular system to walk better. After completing training, a minimal set of stimulators controlled by a mobile device (e.g. smart phone, smart watch) is used for ongoing benefit.

### Augmented Reality

Visual, auditory and sensory stimulus combined with Augmented Reality (AR) (e.g. google glasses, smart phone or other mobile device using the inbuilt camera or future iterations of the technology. One embodiment of our invention claims IP on the use of AR in combination with our stimulating and sensing smart textile network. In AR the user's movements are recorded by the network of sensors or smart textiles and projected into the user's current environment using AR technology. In the AR environment an immersive experience is created with visual and possibly synchronised auditory cues superimposed into the user current environment to (i) improve training effectiveness (ii) help people overcome challenging situations that could reduce movement performance, (iii) enable people to have better control over their movements during. For example, a series of indicators could be superimposed onto the floor (only seen by the user wearing the AR technology) for the user to step over or a series of obstacles to negotiate. Or a virtual trainer could walk beside the user (only seen by the user) demonstrating the proper stepping movements and giving verbal encouragement. The Virtual trainer could highlight the aspects of the movement needing attention (e.g. “take bigger steps” or “knees up”).

### Case Studies

Five people with confirmed diagnoses of Parkinson's disease were invited to test the invention by trying different stimulation locations for the purpose of improving walking ability and control of unwanted lower limb movements. The participants' experiences and perceived improvement in walking ability were recorded. Gait was assessed from the middle 16 meters of a 20 meter walkway using a wearable device attached to the pelvis. Study approval was obtained from the UNSW Human Research Ethics Committee (HC17978). All participants provided informed consent.

### Method

Participants: Participants were recruited from a volunteer database, which includes people with Parkinson's disease who have expressed an interest in participating in research projects. Inclusion criteria were people with a confirmed diagnosis of Parkinson's disease. Exclusion criteria were inability to walk with or without walking aid for 20 meters without stopping and moderate to severe cognitive impairment as defined by a Pfeiffer Short Portable Mental Status Questionnaire (SPMSQ) score <5. Participants were tested while ‘on’ medication.

Peripheral Nerve Stimulation: After completing the medical history questionnaire and signing informed consent participants were fitted with devices to provide ankle or foot arch stimulation to both limbs. They were asked to try walking with the stimulation switched on at the different locations, while the stimulation duty cycle was matched to their preferred cadence and controlled using a phone app. Training and gait assessments were then completed based on using the participant's preferred stimulation location and preferred duty cycle.

Training: Step training synchronised visual cues from a stepping mat with colored targets, auditory cues from a metronome and peripheral nerve stimulation of the ankle or foot arch of both feet. Participants completed 3 by 30 seconds of each stepping exercise at preferred (+0%), slow (−20%) and fast (+20%) cadences. Participant's completed 1 to 3 stepping exercise depending on ability. The stepping exercises progression was: (i) high knees; (ii) forward steps; (iii) backward steps; (iv) side steps; and (v) figure of eight. Walk training synchronised visual cues from stepping targets, auditory cues from a metronome and peripheral nerve stimulation of the ankle or foot arch. Participants walked through three obstacle courses comprising: (i) chairs to be avoided; (ii) colored tiles to step on; and (iii) foam blocks to step over. Participants completed 3 by 30 seconds of each walk exercise at preferred (+0%), slow (−20%) and fast (+20%) cadences.

Gait assessments: Gait assessments were conducted using the middle 16 meters of a 20 meter walkway. Data were collected from a wearable device attached to the pelvis, which recorded the timings of consecutive heel strikes. The times the participant passed the 2-meter and 18-meter marks were marked by a research assistant using a remote trigger. The mean of two walks were recorded for each test. Gait was assessed: (i) at baseline, without stimulation; (ii) with the participants' preferred ankle or foot arch stimulation after training. Gait parameters included walking speed [distance travelled/time taken], step length [distance travelled/steps taken], cadence [steps/minute] and step-time variability [the standard deviation of step times].

User experiences: After each walk participants were asked to rate gait quality and perceived mental effort required to walk using a visual scale from 0%-100%. At the end of the session participants were interviewed about their experiences using a set sequence of questions.

### Results

### Participant 1: HLJ034

Self-reported symptoms: Participant 1 was a 76-year-old male diagnosed with Parkinson's disease for 16 years. Self-reported symptoms included slowness, joint stiffness, walking difficulty, leg weakness and double vision. He reported taking one antiparkinsonian drug in tablet form. He reported being able to walk unaided, but also used a walking frame and wheelchair. He reported frequent freezing of gait (more than once a day) and reported one fall (within the past 6-months) resulting in bruising and grazing but not hospitalisation.

Clinical observations: Participant 1 presented with severe gait impairment. While he was able to walk unaided, he required assistance with postural transitions. His gait was unstable, asymmetric and with a noticeable absences of arm swing. Freezing of gait was observed while turning. During the first session, the participant deteriorated rapidly and required a wheelchair because he was unable to walk after he transitioned from the ‘on’ to the ‘off’ medication state. The participant was invited to attend a second visit, which he completed while ‘on’ medication. Results from the second session are presented. Participant 1's gait visibly improved with stimulation, but some gait impairment remained.

User experiences: Participant 1 enjoyed greatly (5/5) training with peripheral nerve stimulation. He preferred foot arch stimulation over ankle stimulation. Positive comments were that training was “interesting” and the foot arch stimulation “gave a better indication when to move”. Suggestions from improvements included that training “could be more difficult”. When comparing the first walk without stimulation to the last walk with foot arch stimulation, his perceived quality of walking increased by from 50% to 65% while his perceived mental effort required to walk remained unchanged at 50%.

Gait assessments: Baseline gait assessments without stimulation were: speed (94.1 cm/s); step length (51.6 cm); cadence (119.9 steps/min); and step-time variability (50.8 ms). With stimulation, participant 1 walked at a noticeable 20% faster gait speed (112.6 cm/s), with 25% increased step length (61.6 cm), a 3% reduction in step-time-variability (22.7 ms) and a 4% decrease in cadence (117.3 steps/min).

### Participant 2: AZCO31

Self-reported symptoms: Participant 2 was a 56-year-old male diagnosed with Parkinson's disease for 8 years. Self-reported symptoms included loss of sensation to left foot (resulting from a damaged peroneal nerve) inability to dorsiflex the left foot and gait impairment with their left side being most effected. He took 3 antiparkinsonian drugs in tablet form. He reported being able to walk unaided indoors and outdoors. Participant 2 did not report freezing of gait or any falls in the past 12-months.

Clinical observations: Participant 2 presented with mild gait impairment. Although he could walk unaided, he had poor toe clearance and some asymmetry caused by insufficient dorsi-flexion of the left ankle. With stimulation the participant 2 could dorsiflex sufficiently to achieve symmetrical heel strikes with both limbs.

User Experiences: Participant 2 enjoyed greatly (5/5) training with peripheral nerve stimulation. He preferred foot arch stimulation over ankle stimulation. Positive comments were that foot arch stimulation was “magic”, “it works” and is “something that can help people”. Suggestions from improvements included that step training “would benefit from have a mirror placed in front, so can see where stepping without looking down”. When comparing the first walk without stimulation to last walk with foot arch stimulation, his perceived quality of walking increased by from 50% to 80% while his perceived mental effort required to walk reduced from 20% to 0%.

Gait assessments: Baseline gait assessments without stimulation were: speed (124.8 cm/s); step length (78.1 cm); cadence (106.7 steps/min); and step-time variability (18.1 ms). With stimulation, participant 2 walked with 5% faster gait speed (130.6 cm/s), 2% increased step length (80.0 cm), a 9% reduction in step-time-variability (16.4 ms) and a 3% increase in cadence (109.6 steps/min).

### Participant 3: PMF011

Self-reported symptoms: Participant 3 was a 66-year-old female diagnosed with Parkinson's disease for 3 years. Self-reported symptoms included resting tremor and involuntary trembling, joint stiffness and leg pain with their left side being the most effected side. They took 1 antiparkinsonian drug in tablet form. She reported being able to walk unaided indoors and outdoors, but having leg pain including numbness in the left 2^{nd} toe. The participant reported freezing of gait occurring often (once a day) and lasting longer than 30 seconds. The participant reported no falls in the past 12 months, however the freezing episodes caused feelings of insecurity and fear of falling.

Clinical Observations: Participant 3 presented with moderate gait impairment and noticeable resting lower limb tremor (when the limb was unweighted) that was reduced by applying rhythmic stimulus to the lateral malleolus at the ankle, which they liked and commented “could be felt further up the leg”. Although they walked unaided, they walked with short steps and had noticeable gait asymmetry favouring the right limb. Participant 3 responded immediately to rhythmic stimulation of the ankle, which resulted in a marked reduction in lower limb tremor and reduced movement variability.

User Experiences: Participant enjoyed greatly (5/5) training with peripheral nerve stimulation, in particular “liked lifting the knees and taking bigger steps”. She preferred ankle stimulation over foot arch stimulation because she could “feel it more in the legs” and her legs then “didn't shake”. Positive comments included stimulation being “much better than medication”, “helps me not have to think about a thing” and the training “gets me motivated”. However, the app “buttons were not big enough,” which “makes it difficult to navigate”. When comparing the first walk without stimulation to the last walk with ankle stimulation, her perceived quality of walking increased by from 20% to 70% and her perceived mental effort required to walk decreased from 100% to 50%.

Gait assessments: Baseline gait assessments without stimulation were: speed (90.1 cm/s); step length (64.1 cm); cadence (90.1 steps/min); and step-time variability (134.9 ms). With stimulation, participant 3 walked with 23% faster gait speed (110.5 cm/s), 2% increased step length (65.6 cm), an 83% reduction in step-time-variability (22.6 ms) and a 20% increase in cadence (108.3 steps/min).

### Participant 4: MOV071

Self-reported symptoms: Participant 4 was a 72-year-old male diagnosed with Parkinson's disease for 5 years. Self-reported symptoms included global slowness, joint stiffness and loss of balance, with the left side more affected. Other symptoms included leg numbness, weakness and pain. Activities that were particularly difficult to perform included turning over in bed, getting in and out of the car and walking distances over 1 km. He reported taking 4 antiparkinsonian drugs. While he could walk unaided his mobility was affected after a left knee replacement surgery. The participant did not report freezing, however reported 2 falls in the past 12 months (1 in the past 6-months), resulting in bruised fingers, but not hospitalisation.

Clinical Observations: Participant 4 presented with mild to moderate gait impairment. They were able to walk unaided and were physically fit, but had marked variability in their stepping movements. This participant enjoyed the more athletic components of the training program. Participant 4 responded to rhythmic stimulation of the foot arch with increased walking speed and longer step lengths. With stimulation visibly less effort was required to achieve a noticeably more consistent gait pattern.

User Experiences: Participant 4 somewhat enjoyed (4/5) the peripheral nerve stimulation. He found the stimulation was an ‘interestingly different sensation’. He preferred foot arch stimulation over ankle stimulation as the ankle “required far more concentration” whereas the foot arch stimulation provided a “much clearer and a stronger feeling” and “noticeably influenced the pace at which I could move”. Positive comments included that training provided the “ability to pace yourself” and “helps improve balance”. Suggestions for improvements included the “sensors didn't sit comfortably on ankles”, training was “too slow” and “could be more challenging”. When comparing the first walk without stimulation to the last walk with stimulation, perceived walking quality was unchanged at 50% while perceived mental effort required to walk decreased from 50% to 20%.

Gait assessments: Baseline gait assessments without stimulation were: speed (116.7 cm/s); step length (68.1 cm); cadence (109.6 steps/min); and step-time variability (52.1 ms). With stimulation, participant 4 walked with 9% faster gait speed (126.7 cm/s), 7% increased step length (72.9 cm), a 72% reduction in step-time-variability (14.4 ms) and a 3% increase in cadence (113.0 steps/min).

### Participant 5: YBG029

Self-reported symptoms: Participant 5 was a 79-year-old female diagnosed with Parkinson's disease for 14 years. Self-reported symptoms included resting leg tremor, involuntary lower limb trembling, joint stiffness, walking difficulty and loss of balance, with the right side of the body more affected. She reported 2 antiparkinsonian drugs and used a duodopa pump. She used a mobility roller frame both indoors and outdoors. Activities the participant found particularly difficult to perform included walking long distances, walking in public, crossing roads, getting in and out of bed and turning over in bed. The participant experienced arthritis in the right knee, requiring a total knee replacement later this year, resulting in restricted walking and difficulty getting in and out of chairs. The participant did not experience any freezing episodes and had not experienced a fall in the past 12 months.

Clinical Observations: Participant 5 presented with severe mobility impairment and was unable to walk unaided. All tests were completed with a roller frame. She responded immediately to the stimulation. After training, she was able to walk unaided (with peripheral nerve stimulation) and had visibly reduced variability, longer step lengths and increased toe clearances over 20 meters without resting. For consistency, all gait assessments were conducted using the roller.

User experiences: Participant 5 greatly enjoyed (5/5) the peripheral nerve stimulation and preferred foot arch stimulation over ankle stimulation because she ‘could feel it more’. The foot arch stimulation ‘helped me to walk’, giving her ‘more of a purpose’. Positive comments included the experience was ‘very inspiring’, ‘I felt I was doing something really good and I achieved something I didn't think I could normally do’ and she was ‘more than happy’. Suggestions for improvements included that the training app was ‘a bit confusing’. When comparing the first walk without stimulation to the last walk with stimulation, her perceived quality of walking increased from 30% to 60% and her perceived mental effort required to walk decreased from 40% to 20%.

Gait assessments: Gait assessments were conducted with the aid of a roller. Baseline gait assessments without stimulation were: speed (68.4 cm/s); step length (39.0 cm); cadence (110.2 steps/min); and step-time variability (30.0 ms). With stimulation, participant 5 walked with 17% faster gait speed (80.3 cm/s), 17% increased step length (45.7 cm), a 3% reduction in step-time variability (29.0 ms) and a 1% increase in cadence (111.8 steps/min).

### Clinical Study—Long-Term Effects

Methods: Eleven people with Parkinson's disease undertook a 2-week intervention that used vibratory stimuli while completing a walking program. Participants had their gait (spatiotemporal, step time variability and gait stability) assessed at baseline and reassessment. Participants were provided with a choice of ankle stimulation using a sock form factor or foot arch stimulation using either an insole or sock form factor. The stimulation duty cycle was matched to each participant's preferred cadence. During the walking program, both left and right limbs stimulated together.

Participants were aged 46 to 82 years (mean 66.8 years), 7 male and 4 female with a mean Parkinson's disease duration of 10.3 years. Participants reported falling between 0 and 13 times in the previous month (mean 2.1 falls/month).

Results: Comparing gait at baseline without stimulation to gait at reassessment also without stimulation significant (p<0.05) improvements were observed including a 19% increase in gait speed from 1.04 m/s to 1.24 m/s and 32% decrease in step-time-variability from 44.1 ms to 29.8 ms (see Table 1 below).

Results table: Comparison of significantly improved gait measures during walking without stimulation at baseline and 2-week reassessment. Data are mean (SD).

TABLE 1

| | Baseline | Reassessment | P |
|---|---|---|---|
| Gait speed (m/s) | 1.04 (0.37) | 1.24 (0.46) | 0.004 |
| Cadence (steps/min) | 105 (13) | 114 (9) | 0.026 |
| Step length (cm) | 64.8 (18.7) | 71.7 (25.7) | 0.032 |
| Step time variability (ms) | 44.1 (34.6) | 29.8 (23.9) | 0.010 |

Conclusion: Stimulation combined with walk training results in improvements in walking ability that persist even after stimulation is ceased. This is in addition to the immediate effects of stimulation on walking described in the case studies above.

### Interpretation

Reference throughout this specification to “one embodiment”, “some embodiments” or “an embodiment” means that a particular feature, structure or characteristic described in connection with the embodiment is included in at least one embodiment of the present disclosure. Thus, appearances of the phrases “in one embodiment”, “in some embodiments” or “in an embodiment” in various places throughout this specification are not necessarily all referring to the same embodiment. Furthermore, the particular features, structures or characteristics may be combined in any suitable manner, as would be apparent to one of ordinary skill in the art from this disclosure, in one or more embodiments.

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

It should be appreciated that in the above description of exemplary embodiments of the disclosure, various features of the disclosure are sometimes grouped together in a single embodiment, Figure, or description thereof for the purpose of streamlining the disclosure and aiding in the understanding of one or more of the various inventive aspects. This method of disclosure, however, is not to be interpreted as reflecting an intention that the claims require more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this disclosure.

Furthermore, while some embodiments described herein include some but not other features included in other embodiments, combinations of features of different embodiments are meant to be within the scope of the disclosure, and form different embodiments, as would be understood by those skilled in the art. For example, in the following claims, any of the claimed embodiments can be used in any combination.

In the description provided herein, numerous specific details are set forth. However, it is understood that embodiments of the disclosure may be practiced without these specific details. In other instances, well-known methods, structures and techniques have not been shown in detail in order not to obscure an understanding of this description.

Thus, while there has been described what are believed to be the preferred embodiments of the disclosure, those skilled in the art will recognise that other and further modifications may be made thereto without departing from the spirit of the disclosure, and it is intended to claim all such changes and modifications as fall within the scope of the disclosure.

1. A stimulation device for guiding movement of at least part of a body, the device comprising:
a plurality of body-fixed stimulators, configured to provide stimulus to the part of the body;
at least one control unit in communication with the at least one body-fixed stimulator, the control unit configured to generate a control signal to control the at least one body-fixed stimulator;
at least one sensor in communication with the at least one control unit, the at least one sensor configured to receive monitoring data; and
wherein the control signal is generated on the basis of the monitoring data.

2. A stimulation device according to claim 1 wherein the plurality of stimulators are cylindrical vibrational motors or coin vibrational motors.

3. A stimulation device according to claim 1 wherein at least one stimulator is securely disposed adjacent to at least one layer of material to fix the location of the stimulator relative to the body.

4. A stimulation device according to claim 1, wherein each of the plurality of body-fixed stimulators are disposed in a predefined location relative to the part of the body.

5. A stimulation device according to claim 1, wherein the control signal is generated when at least one pre-defined criteria is met.

6. A stimulation according to claim 1, wherein the control signal controls timing, synchronisation, location and intensity to coordinate the stimulation provided by the plurality of stimulators.

7. A stimulation device according to claim 5, wherein the control signal is optimised based on the monitoring data to iteratively adjust control of the plurality of stimulators in real time to guide movement.

8. A stimulation device according to claim 1, wherein the control unit is configured to:
a) continuously receive monitoring data from the at least one sensor;
b) generate an adjusted control signal to adjust the intensity and/or pattern of stimulation provided by the plurality of stimulators based on the received monitoring data;
c) receive updated monitoring data from the at least one sensor;
d) compare at least one parameter relating to a pre-defined criteria for a target movement to the updated monitoring data;
wherein if the updated monitoring data meets the pre-defined criteria for a target movement, the control unit is configured to stop stimulation provided by the plurality of stimulators; and
wherein if the updated monitoring data does not meet the pre-defined criteria for a target movement, the control unit is configured to repeat steps b to d so as to iteratively optimise the adjusted control signal until a target movement is achieved.

9. A stimulation device according to claim 1 wherein the device comprises a plurality of sensors.

10. A stimulation device according to claim 1 wherein the at least one sensor includes a gyroscope.

11. A stimulation device according to claim 1, wherein guiding movement of at least part of a body includes directing the part of the body to achieve a target movement or position.

12. A stimulation device according to claim 1, wherein guiding movement includes directing the body to increase desirable movement and/or decrease undesirable movement.

13. A stimulation device according to claim 11, wherein the target movement or position is stable gait.

14. A stimulation device according to claim 11, wherein the target movement or position is reduced limb tremor.

15. A stimulation device according to claim 1 wherein the at least one sensor is fixed to the body by way of integration with a textile.

16. A stimulation device according to claim 15, wherein the textile forms at least part of a wearable garment.

17. A stimulation device according to claim 16 wherein the wearable garment is in the form of a sock.

18. A method of guiding movement of at least part of a body comprising the steps of:
(a) comparing at least one parameter relating to a movement with at least one pre-defined criteria;
(b) generating a control signal from at least one control unit if the at least one parameter does not meet the pre-defined criteria; and
(c) providing a stimulus to the body in response to the control signal through at least one body-fixed stimulator to guide the movement.

19. (canceled)

20. A method of guiding movement of at least part of a body comprising the steps of:
(a) providing a stimulus to the body through at least one body-fixed stimulator to guide a movement;
(b) receiving monitoring data related to at least one parameter relating to a target movement from at least one sensor;
(c) comparing the at least one parameter relating to the target movement with at least one pre-defined criteria;
(d) providing an adjusted stimulus to the body through a generated control signal if the at least one parameter does not meet the pre-defined criteria, wherein the adjusted stimulus is generated based on monitoring data and historical data; and
(e) ceasing stimulation if the parameter meets the pre-defined criteria.

21. A method according to claim 20, further comprising the steps of:
updating at least one stimulation setting based on the received monitoring data and the target movement to define at least one updated stimulation setting; and
generating an optimised control signal with the updated stimulation setting to provide an adjusted stimulus to the body to guide the body towards achieving a target movement.

22.-34. (canceled)
